(19) United States
(12) Patent Application Publication
(10) Pub. No.: US 2024/0252456 A1
(43) Pub. Date: Aug. 1, 2024

(54) ORAL COMPOSITION, SKIN COSMETIC AND HAIR COSMETIC

(71) Applicant: MARUZEN PHARMACEUTICALS CO., LTD., Hiroshima (JP)

(72) Inventors: Yosuke NISHITANI, Hiroshima (JP); Shota HAYASHI, Hiroshima (JP); Miyuki KOMOTO, Hiroshima (JP)

(21) Appl. No.: 18/560,211

(22) PCT Filed: May 12, 2022
(86) PCT No.: PCT/JP2022/020078

§ 371 Date: Nov. 10, 2023

(30) Foreign Application Priority Data
May 12, 2021 (JP) 2021-081310

(51) Int. Cl. A61K 31/192; A61K 8/36; A61K 9/00; A61P 3/00; A61Q 7/00; A61Q 19/02; A61Q 19/08

(52) U.S. Cl. CPC A61K 31/192; A61K 8/36; A61K 9/0053; A61P 3/00; A61Q 7/00; A61Q 19/02; A61Q 19/08; A61K 2800/92

(57) ABSTRACT

Compounds 1 to 3 represented by General Formula (I) below are used as the active ingredients of the anti-metabolic syndrome agent, skin whitening agent, anti-aging agent, hair growth agent, anti-inflammatory agent, and liver function-improving agent of the present invention. The oral composition, skin cosmetic, and hair cosmetic of the present invention are compounded with compounds 1 to 3 represented by General Formula (I) below. The present invention can provide an anti-metabolic syndrome agent, a skin whitening agent, an anti-aging agent, a hair growth agent, an anti-inflammatory agent, and a liver function-improving agent that have excellent actions and effects, and can also provide an oral composition, a skin cosmetic, and a hair cosmetic that are suitable for an anti-metabolic syndrome application, a skin whitening application, an anti-aging application, a hair growth application, an anti-inflammatory application, and a liver function-improving application.

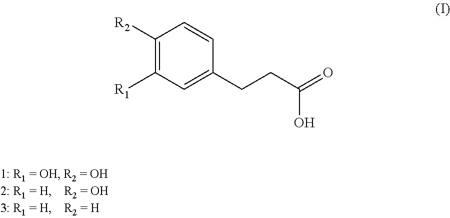

### TECHNICAL FIELD

The present invention relates to an anti-metabolic syndrome agent, a skin whitening agent, an anti-aging agent, a hair growth agent, an anti-inflammatory agent, and a liver function-improving agent that contain compounds derived from natural products as active ingredients. Furthermore, the present invention also relates to an oral composition, a skin cosmetic, and a hair cosmetic that are compounded with such compounds.

### BACKGROUND ART

In recent years, lifestyle habits such as overeating and lack of exercise have caused an increase in body fat and obesity. Such an increase in obesity is observed not only in humans but also in pets and farm animals. Obesity causes metabolic syndromes such as hyperlipidemia and arteriosclerosis and is therefore not only a problem in terms of beauty, but also a serious problem in terms of health.

Here, cyclic AMP (cAMP) is known to be involved in lipolysis in vivo. The cAMP activates lipase in vivo, and fat is decomposed into fatty acids and glycerol by the activated lipase. However, activation of cAMP phosphodiesterase induces decomposition of cAMP and inhibits the activation of lipase. It is therefore considered that the amount of cAMP in cells can be increased to promote the decomposition of fat by inhibiting the activity of cAMP phosphodiesterase.

It is also known that platelet aggregation, which causes an inflammatory reaction, is related to the concentration of cyclic AMP (cAMP) in platelets, and that when cAMP is decomposed by cAMP phosphodiesterase and the concentration of cAMP decreases, platelets become more likely to aggregate. It is therefore considered that if the action of cAMP phosphodiesterase is suppressed to prevent a decrease in the cAMP concentration, the platelet aggregation can be avoided, thereby preventing, treating, or ameliorating allergic diseases, inflammatory diseases, etc. Tubeimoside I and the like are known to have a cAMP phosphodiesterase activity inhibitory action (see Patent Document 1).

Dipeptidyl peptidase IV (also referred to as “DPP IV,” hereinafter) is one of serine proteases and has an enzyme activity that recognizes the second proline or alanine from the N-terminus and cleaves its C-terminus side. DPP IV is expressed on the cell surfaces of epithelial and endothelial cells of tissues such as the kidney, liver, intestinal canal, and placenta and T cells, and is considered to be involved in various physiological phenomena through its enzymatic activity and the like.

Examples of substrates for DPP IV include hormones called incretins. Incretin is a general term for hormones that are secreted from the intestinal canal due to stimulation by nutrients and promote insulin secretion from pancreatic B cells depending on the blood sugar, and GLP-1, GIP, etc. are known. These incretins have actions of not only promoting blood sugar-dependent insulin secretion, but also suppressing glucagon secretion from a cells, lowering blood pressure, suppressing gastric emptying, also suppressing food intake by acting on the hypothalamus, etc. (see Non-Patent Document 1). It is, however, known that incretins are decomposed by DPP IV, so the half-life of GLP-1 in vivo is, for example, about 1.5 minutes. It is therefore expected that if the enzymatic activity of DPP IV can be inhibited, the half-life of incretins in vivo can be extended, and the incretins are thereby useful in treating metabolic syndromes such as the type 2 diabetes, obesity, hypertension, and insulin resistance through the aforementioned actions of incretins.

DPP IV is the same as CD26, which is one of the T cell activation markers, and is known to use many immunoregulatory peptides as substrates and control their activities. It is therefore considered that by controlling the activity of DPP IV, it is possible to control autoimmune diseases such as rheumatoid arthritis and immune reactions such as transplant rejection. Furthermore, DPP IV is known to be involved in the metabolism of several neuropeptides and growth hormones; infiltration, metastasis, angiogenesis, and the like in cancer; infection of lymphocytes by HIV, etc. It is thus believed that inhibition of the activity of DPP IV enables treatment of neurological disorders such as algia, neurodegenerative diseases, and neuropsychiatric diseases (e.g. sciatica, Alzheimer's disease, depression, etc.); growth hormone deficiency and diseases for which growth hormone is used for treatment; cancer (e.g., T-cell lymphoma, acute lymphoblastic leukemia, thyroid cancer, basal cell carcinoma, breast cancer, etc.); HIV infection (AIDS), etc.

Melanin in the skin plays a role in protecting living organisms from ultraviolet rays, but the excessive formation and uneven accumulation may cause darkening and age spots on the skin. It is generally said that melanin is formed through the action of the enzyme tyrosinase, which is biosynthesized in pigment cells, from tyrosine to dopa, from dopa to dopaquinone, and then through intermediates such as 5,6-dihydroxyindophenol. To prevent, treat, or ameliorate dark skin (skin pigmentation), age spots, freckles, etc., therefore, it is considered to inhibit the activity of tyrosinase, which is involved in melanin production, or to suppress melanin production.

Conventionally, to prevent, treat, or ameliorate skin pigmentation, age spots, freckles, etc., treatments have been performed by externally applying skin whitening agents whose active ingredients are chemically synthesized products such as hydroquinone. Chemically synthesized products such as hydroquinone, however, may have side effects such as skin irritation and allergies. There is therefore a desire to develop skin whitening agents whose active ingredients are highly safe natural raw materials. For example, polygonum hydropiper extracts (see Patent Document 2) and the like are known as skin whitening agents having tyrosinase activity inhibitory actions. There are also known, for example, extracts from a plant of the genus Saussurea (see Patent Document 3) and the like that have melanin production suppressive actions.

The epidermis and dermis of the skin are composed of epidermal cells, fibroblasts, and extracellular matrices such as collagen, elastin, and hyaluronic acid that exist outside these cells and support the skin structure. In young skin, fibroblasts proliferate actively, and the interaction between skin tissues such as fibroblasts and extracellular matrix components maintains homeostatic properties thereby to ensure moisture retention, flexibility, elasticity, etc., and the skin remains in a fresh state with a firm and glossy appearance.

However, the influence of certain external factors such as ultraviolet irradiation, extremely dry air, and excessive skin cleansing or the progression of aging reduces the production amount of collagen, elastin, and hyaluronic acid, which are the main components of the extracellular matrices, and causes their decomposition and degradation. This results in an decrease in the moisturizing function and elasticity of the skin, and abnormal exfoliation of cuticles occurs, so the skin loses its firmness and luster and exhibits aging symptoms such as rough skin and wrinkles. Thus, the decrease/denaturation of matrix components such as collagen, elastin, and hyaluronic acid is involved in the changes due to skin aging, that is, the wrinkles, dullness, changes in texture, decreased elasticity, etc. Therefore, promoting the production of collagen, elastin, or hyaluronic acid is important in preventing, treating, or ameliorating the skin aging.

Here, among these extracellular matrix components, collagen is a fibrous protein that contributes to maintaining the structure and mechanical strength of skin tissues. If the production of collagen can be promoted, the wrinkles and sagging will be less likely to occur, and it is believed that aging symptoms of the skin, such as coarse texture, loss of firmness, and decrease in elasticity, can be prevented, treated, or ameliorated.

It is also known that collagen is present in large amounts in bones, tendons, ligaments, corneas, blood vessels, etc., and a decrease in the collagen production due to aging or the like is a cause of osteoporosis, etc. Furthermore, it is known that the production amount of collagen is increased during the wound healing process and the collagen serves as a scaffold for fibroblasts, etc. thereby to promote the wound healing. Therefore, promoting the collagen production is important from the viewpoint of preventing or treating osteoporosis and the like and promoting wound healing. As substances having collagen production promotive actions, for example, extracts from kamala (see Patent Document 4) and the like are known.

On the other hand, among the aforementioned extracellular matrix components, elastin is a fiber that gives elasticity to the skin tissue. If the production of elastin can be promoted, the wrinkles and sagging will be less likely to occur, and it is believed that aging symptoms of the skin, such as loss of firmness and decrease in elasticity, can be prevented, treated, or ameliorated.

Furthermore, elastin is decomposed by an enzyme called elastase, and elastase is activated by irradiation with ultraviolet rays, thereby accelerating the decomposition of elastin. It is therefore considered that by inhibiting the activity of elastase, the decomposition of elastin is suppressed, and it is possible to prevent/ameliorate the skin aging symptoms such as loss of firmness and decrease in elasticity.

Furthermore, elastin is widely expressed in tissues that require elasticity in the body, such as the lungs and blood vessels, in addition to skin tissues. It is known that if normal elastin decreases in these tissues as aging progresses, the elasticity of lungs, blood vessels, etc. decreases, thus causing pulmonary diseases such as emphysema and vascular diseases such as high blood pressure and aneurysms. It is also known that smoking and the like increase the activity of elastase in vivo, and it is considered that this may destroy the alveolar walls and lead to emphysema, etc. It is also considered that the increased activity of elastase may destroy pulmonary capillaries, leading to acute respiratory distress syndrome (ARDS) such as pulmonary edema.

Therefore, if the production of elastin can be promoted, the decline in elasticity in the lungs, blood vessels, etc. will be less likely to occur, and it is believed that pulmonary diseases such as emphysema, vascular diseases such as high blood pressure and aneurysms, etc. can be prevented/treated. It is also believed that if the activity of elastase in vivo can be inhibited, diseases of respiratory system, such as emphysema and pulmonary edema can be prevented/treated.

Extracts from plants belonging to the genus Hippophae L., for example, are known as having elastin production promotive actions (see Patent Document 5). Furthermore, for example, fruit extracts of star fruit and the like are known as having elastase activity inhibitory action (see Patent Document 6).

On the other hand, among the aforementioned extracellular matrix components, the hyaluronic acid, which is a type of mucopolysaccharide, has a function of retaining cells by filling the intercellular spaces and also has a variety of functions of retaining water in the intercellular spaces, giving lubricity and flexibility to tissues, resisting external force such as mechanical disturbances, etc. It is believed that if the production of hyaluronic acid can be promoted, it will be possible to prevent, treat, or ameliorate skin aging symptoms such as rough skin, wrinkles, dullness, changes in texture, decreased elasticity, and decreased moisturizing function. It is also believed that skin aging can be prevented, treated, or ameliorated by promoting the expression of hyaluronic acid synthase 3 (HAS3), which is involved in promoting the synthesis of epidermal hyaluronic acid.

Hyaluronic acid is present not only in skin tissues but also in cartilage, joint fluid, umbilical cord, vitreous body of the eye, and other connective tissues. Among these, the hyaluronic acid contained in synovial fluid coats the surface of articular cartilage and is useful for the smooth operation of joints due to its lubricating function, coating/protecting function for cartilage, etc. On the other hand, it is known that the concentration of hyaluronic acid in joint fluid decreases in arthritis such as chronic rheumatoid arthritis. It is therefore considered that by promoting the production of hyaluronic acid, the arthritis such as chronic rheumatoid arthritis, arthritis deformans, suppurative arthritis, gouty arthritis, traumatic arthritis, or osteoarthritis can be prevented or treated. It is also known that during the healing process of a wound or burn, granulation (tissue) is formed while hyaluronic acid increases significantly in the granulation. It is therefore considered that by promoting the production of hyaluronic acid, the healing of wounds or burns can be promoted. As substances having hyaluronic acid production promotive actions, extracts from kamala (see the aforementioned Patent Document 4) and the like are known. In addition, licorice leaf extracts (see Patent Document 7) and the like are known as having hyaluronic acid synthase 3 (HAS3) expression promotive actions.

On the other hand, a basement membrane exists at the boundary between the epidermis and dermis that constitute the skin. The basement membrane not only connects the epidermis and dermis, but also plays an important role in maintaining skin functions (see Non-Patent Document 2). The main framework of the basement membrane has a network structure composed of type IV collagen. Various glycoproteins whose main component is laminin-332 exist at the boundary between the basement membrane and the epidermis and connect the basement membrane and the epidermis, and such laminin-332 is produced by epidermal keratinocytes present in the epidermis. In young skin, the interaction between the epidermis and dermis maintains homeostatic properties through the action of the basement membrane thereby to ensure moisture retention, flexibility, elasticity, etc., and the skin remains in a fresh state with a firm and glossy appearance.

However, the influence of certain external factors such as ultraviolet irradiation, extremely dry air, and excessive skin cleansing or the progression of aging causes decomposition/degradation of the laminin-332, which is the main component of the basement membrane, and the basement membrane structure is destroyed (see Non-Patent Document 3). This results in an decrease in the moisturizing function and elasticity of the skin, and the cuticles start abnormal exfoliation, so the skin loses its firmness and luster and exhibits aging symptoms such as rough skin and wrinkles. Thus, a decrease in the basement membrane components and structural changes of the basement membrane are involved in the changes due to skin aging, that is, the wrinkles, dullness, changes in texture, decreased elasticity, etc., and it is considered that the skin aging symptoms can be prevented/ameliorated by promoting production of the laminin-332.

Laminin is composed of various combinations of α, β, and γ chains, and currently 15 types (laminin 1 to laminin 15) are known. Among these, laminin-332 (α3β3γ2) is present in large amounts in the basement membranes of epithelial tissues such as the skin, digestive organs, kidney, and lungs. Genetic diseases caused by congenital abnormalities in the genes encoding each chain of laminin-332 (Herlitz junctional epidermolysis bullosa) is known to exhibit fatal symptoms in which the epidermis exfoliates all over the body. Laminin-332 is also known to strongly adhere to cells (high cell adhesion activity) and strongly promote cell motion (high cell motion activity) compared to other extracellular matrix molecules.

Thus, laminin-332 is known to promote cell motion in the damaged skin and promote wound healing due to its high cell motion activity (see Patent Document 8). That is, promoting the production of laminin-332 is important in promoting healing of skin injuries in which the basement membrane structure is destroyed.

The epidermis has functions of alleviating external stimuli and controlling the loss of internal components such as water and is composed of a four-layered structure that starts from the basal layer, which is the lowest layer, to the spinous layer, the granular layer, and the stratum corneum. Most cells present in each layer are keratinocytes differentiated from the basal layer. Keratinocytes divide and proliferate in the basal layer, differentiate while passing through the spinous layer and the granular layer to become corneocytes, form the stratum corneum, which is composed of keratin protein fibers with strong crosslinks, and eventually fall off from the stratum corneum as scurf.

The stratum corneum exists in the outermost layer of the skin and serves as a physical barrier against external stimuli. To achieve this barrier function, the skin undergoes a cycle (keratinization) from when keratinocytes are produced in the basal layer until they become scurf and fall off, and this cycle is usually repeated every four weeks to metabolize the epidermis. However, the metabolic function of this stratum corneum also declines with age, leading to skin problems such as stiffness, dullness, pigmentation, and rough skin. It is thus considered that by promoting the proliferation of keratinocytes and restoring the skin's metabolic function, it is possible to ameliorate the skin aging such as stiffness, dullness, and pigmentation. Earth shell extracts (see Patent Document 9) and the like are conventionally known as substances having epidermal keratinocyte proliferation promotive action.

To promote cell proliferation, it is important to supply cells with the energy necessary for cell division. ATP is an example of an energy substance in living organisms, and it is considered that increasing the production amount of ATP promotes the intracellular energy metabolism and leads to cell proliferation. As suggested above, however, it has been reported that the amount of ATP, which is an energy substance, is reduced in cells with decreased function or aged cells compared to normal cells (see Patent Document 10).

Thus, it is considered that if the production of ATP in cells can be promoted, the cells can be activated to promote the cell division, and the proliferation ability of the cells can be restored. In particular, promoting the ATP production in skin cells is important in promoting skin turnover, restoring the skin's metabolic function, and preventing/ameliorating the skin aging such as wrinkles, dullness, and loss of texture. Conventionally, glycogen (see Patent Document 10), extracts from natural products such as peach (see Patent Document 11), and the like are known as having ATP production promotive actions.

Glutathione is a tripeptide composed of three amino acids: glutamic acid, cysteine, and glycine, and is a compound having cysteine residues, which are the main intracellular residues. Glutathione in cells functions to trap radicals, regulate cell functions through redox, metabolize foreign substances, and serve as an SH donor for various enzymes, and is also known as an antioxidant component against active oxygen and the like. Its expression of actions is considered to be derived from cysteine residues. However, it has been reported that the amount of glutathione in cells becomes deficient or decreased due to excessive oxidative stress, addition of foreign substances, aging, etc., and this reduces the ability of cells to defend against the oxidative stress and is considered to be one of the causes of damage to structural components such as DNA and protein of cells.

Diseases in which a decrease or deficiency in the amount of intracellular glutathione is known to be involved with pathological conditions include a group of diseases induced by oxidative stress such as the formation of age spots on the skin and diseases in the liver (caused by excessive alcohol consumption or ingestion of foreign substances such as heavy metals and chemicals). That is, it is considered that promoting the production of glutathione increases the ability of cells to defend against oxidative stress and can prevent/treat the above group of diseases caused by a decrease or deficiency in the amount of glutathione in cells. Liquiritigenin (see Patent Document 12) and the like are known as having glutathione production promotive actions.

Among the stratum basale, stratum spinosum, stratum granulosum, and stratum corneum that constitute the epidermis, particularly in the stratum granulosum, the cell membrane thickens to form a thickened cell membrane, and the action of transglutaminase-1 subjects protein molecules to glutamyl-lysine crosslink to form strong keratin protein fibers. Furthermore, ceramide or the like is covalently bonded to a part of that to take a hydrophobic structure, which provides the foundation for the lamellar structure of intercellular lipids, thus forming the basis of the stratum corneum barrier function.

However, if the production amount of transglutaminase-1 in the epidermis decreases as the aging progresses, the stratum corneum barrier function and the skin moisturizing function will deteriorate, resulting in the skin aging symptoms such as rough skin and dry skin and the development of dry skin diseases (e.g., atopic dermatitis, psoriasis, ichthyosis, and the like). It is therefore considered that by promoting the production of transglutaminase-1 in the epidermis, the skin aging symptoms, dry skin diseases, etc. can be prevented, treated, or ameliorated. Extracts from Hunan sweet tea (see Patent Document 13) and the like are known as having transglutaminase-1 expression promotive actions.

Ceramide is produced during the keratinization process of epidermal cells by the actions of enzymes including serine palmitoyltransferase (SPT), which is known as the rate-determining enzyme for ceramide synthesis, based on serine and palmitoyl-CoA. Ceramide exists specifically as the main component of intercorneocyte lipids that cover the outermost layer of the skin, and plays an important role in maintaining the skin's inherent function as a barrier membrane between the living body and the outside world.

The structure of the stratum corneum is taken as an analogy to bricks and mortar, with about 15 layers of corneocytes stacked together by intercellular lipids to form a strong barrier membrane. The corneocytes retain moisture by containing natural moisturizing factors whose main components are amino acids, while the intercorneocyte lipids contain approximately 50% ceramide as the main component and are composed of amphipathic lipids such as cholesterol and fatty acids. The intercorneocyte lipids are thus characterized by a so-called lamellar structure in which hydrophobic portions and hydrophilic portions are alternately repeated.

Deterioration of the skin's barrier function due to various internal/external factors increases the transepidermal water loss, which causes skin dryness, desquamation, itching, etc., resulting in so-called dry skin. Furthermore, deterioration of the skin's barrier function increases skin inflammation, leading to a vicious cycle in which the defense function against various stimuli from the outside world decreases. Recent studies have reported a decrease in cuticle ceramide components (so-called intercellular lipids) and changes in their composition due to aging or in patients with atopic dermatitis, which is known as a barrier disorder (see Non-Patent Document 4). It is thus becoming widely known that ceramides are important in maintaining and ameliorating the barrier function of the skin. As methods for ameliorating the barrier function of the skin, there are known methods such as supplementing ceramide from the outside (see Non-Patent Document 5) and increasing the ceramide production ability within the skin (see Non-Patent Document 6).

It is known that in skin cells, aquaporins, known as water channels, are expressed on the cell membrane and play a role in taking water and other low-molecular substances from the intercellular spaces into the cells. In humans, 13 types of aquaporins (AQP0 to AQP12) are known to exist. AQP3 is mainly present in epidermal cells, and is considered to play a role in taking in not only water but also low-molecular-weight compounds such as glycerol and urea that are involved in a moisture retention action.

However, AQP3 decreases with age, and it has been suggested that this is a factor in the decline in the moisture retention function, so it is considered that by promoting the expression of AQP3, the moisture retention function, barrier function, etc. depending on the aging can be controlled (see Non-Patent Document 7). For example, extracts from leaves of star fruit (see Patent Document 14) and the like are known as having AQP3 expression promotive actions.

Filaggrin is a component of the skin and it is considered that filaggrin is involved in the barrier function of the skin and has a function of preventing the invasion of allergens, toxins, and infectious organisms. It is known that decreased function of the filaggrin due to genetic mutations is associated with the risk of developing atopic diseases, including atopic dermatitis (such as eczema, skin inflammation, and skin itching), allergies, asthma, etc., leading to skin diseases such as ichthyosis vulgaris in more severe cases (see Non-Patent Document 8).

On the other hand, amino acids, which are the main components of Natural Moisturizing Factors (NMF), are produced by the decomposition of filaggrin derived from keratohyalin granules within the stratum corneum. This filaggrin is expressed as profilaggrin in epidermal keratinocytes present in the granular layer just below the stratum corneum. It is known that the profilaggrin is then immediately phosphorylated, accumulated in keratohyalin granules, dephosphorylated, hydrolyzed, and decomposed into filaggrin, and migrates to the stratum corneum, increasing the aggregation efficiency of keratin filaments and participating in the internal structure of corneocytes (see Non-Patent Document 9). In recent years, it has been known that filaggrin is extremely important and essential for retaining moisture in the skin and the synthetic ability of filaggrin deteriorates depending on conditions such as dryness to reduce the amount of amino acids in the stratum corneum (See Non-Patent Document 10).

It is therefore considered that by promoting the expression of filaggrin (profilaggrin) in epidermal keratinocytes, it is possible to prevent/treat or ameliorate atopic diseases including atopic dermatitis (such as eczema, skin inflammation, and skin itching), allergies, asthma, etc. Furthermore, by promoting the expression of filaggrin and thereby increasing the amount of amino acids in the stratum corneum, it is expected that the water environment of the stratum corneum can be essentially ameliorated. Extracts from Artemisia princeps (see Patent Document 15) and the like are known as substances having filaggrin expression promotive actions.

Conventionally, it has been considered that only the stratum corneum is responsible for the barrier function of the skin, but in recent years, it has been discovered that the skin's barrier function collapses when the constituent proteins of tight junctions (which may be abbreviated as “TJ,” hereinafter) present in the granular layer of the epidermis are deleted at the genetic level, and it is considered that the TJ also plays an important role in the barrier function of the skin (see Non-Patent Document 11). The TJ is an intercellular adhesion structure that not only brings adjacent cells into close contact with each other, but also controls the permeation of substances by sealing gaps between cells. The TJ is composed of cell membrane proteins such as claudin and occludin, lining proteins such as ZO-1 and ZO-2, etc., and these proteins are considered to constitute the framework of TJ strands and control the barrier function of the TJ (see Non-Patent Document 12). Here, it is considered that if the expression of claudin or occludin decreases for some reason, the TJ will be structurally destroyed and no longer function as a permeation barrier for substances, thereby causing skin symptoms such as dry skin, rough skin, atopic dermatitis, and various infections.

It is therefore considered that by promoting the production of claudin and occludin in the epidermis to accelerate the TJ formation in epidermal keratinocytes, the barrier function and moisture retention function of the skin are enhanced to prevent or ameliorate the skin symptoms such as dry skin, rough skin, atopic dermatitis, and various infections. Aspalathus linearis extracts (Patent Document 16) and the like are known as having claudin production promotive actions and occludin production promotive actions.

Carbohydrates are extremely important as an energy source in living organisms including humans. On the other hand, however, carbohydrates are known to cause a glycation reaction with proteins. The glycation reaction is a series of reactions in which the first step is a non-enzymatic reaction between the carbonyl group of a carbohydrate and the amino group of a protein, etc., and advanced glycation end-products (which may be referred to as “AGEs,” hereinafter) are finally formed from a Schiff base through an Amadori compound. Due to the glycation reaction, proteins are non-enzymatically modified with sugars, which causes denaturation of proteins, crosslinking between proteins, etc., resulting in deterioration of functions of proteins.

The glycation reaction not only causes direct damage by modifying/structurally changing extracellular matrix constituent proteins such as collagen, but also has other effects such as triggering cellular responses when recognized by receptors whose ligands are glycated proteins. In particular, the effects of the glycation reaction are serious for diabetic patients with high blood glucose levels. It is known that protein glycation is a contributory factor to diabetic complications such as diabetic neuropathy, diabetic retinopathy, and diabetic nephropathy. It is also known that the glycation reaction in blood vessel walls leads to the progression of arteriosclerosis due to damage to endothelial cells and accumulation of denatured proteins. Furthermore, extracellular matrix components including collagen account for the majority of the dry weight of tissues such as bone and skin. Therefore, for example, when collagen is glycated into a state of being abnormally crosslinked, osteoporosis, osteoarthritis, or the like develops in bones and cartilage tissues, and dullness or the like due to decreased elasticity, yellowing, etc. occurs in the skin. Furthermore, the abnormally crosslinked collagen or the like is less susceptible to decomposition by collagenase, etc., and there is therefore a problem in that the expression of collagenase or the like is induced to decompose even normal collagen.

Thus, if the glycation reaction can be suppressed in some way, for example by suppressing the formation of AGEs or promoting the decomposition of AGEs, it will be useful to prevent or treat the aforementioned diseases, such as diabetic complications, arteriosclerosis, osteoporosis, and osteoarthritis. Furthermore, it is expected to be effective in preventing or ameliorating the skin elasticity deterioration, dullness, etc. Osmanthus extracts (see Patent Document 17) and the like are known as having AGEs formation inhibitory actions and AGEs decomposition promotive actions.

Most steroid hormones are in molecular forms secreted from producing organs and bind to receptors to express their actions. However, in the case of male hormones collectively called androgens, for example, testosterone enters the cells of target organ, binds to a receptor after being reduced to 5α-dihydrotestosterone (5α-DHT) by 5α-reductase, and expresses the action as androgen.

Androgens are important hormones, but when they act excessively, they may induce a variety of undesirable symptoms such as male pattern alopecia, hirsutism, seborrhea, acne (comedo or the like), benign prostatic hypertrophy, prostate tumors, and premature male puberty. In order to ameliorate these various symptoms, there has conventionally be known a method of suppressing the action of excess androgen, specifically, a method of suppressing the production of active 5α-DHT by inhibiting the action of testosterone 5α-reductase, which reduces testosterone to active 5α-DHT. Hitherto, for example, extracts from Elsholtzia bodinieri (see Patent Document 18) and the like are known as having testosterone 5α-reductase inhibitory actions.

Hair repeats growth and defluxion according to a cyclical hair cycle (hair growth cycle) consisting of an anagen phase, a catagen phase, and a telogen phase. In this hair cycle, the stage from the telogen phase to the anagen phase, in which new hair follicles are formed, is considered to be the most important for hair growth, and follicle dermal papilla cells are considered to play an important role in the proliferation/differentiation of hair follicle epithelial cells at that stage. The follicle dermal papilla cells are cells that are present inside the hair follicle epithelial cells composed of outer root sheath cells and matrix cells near the hair root and that are located in the hair root base portion surrounded by a basement membrane, and play an important role in the proliferation/differentiation of hair follicle epithelial cells and the formation of hair, such as acting on the hair follicle epithelial cells to promote their proliferation (see Non-Patent Document 13).

Thus, the follicle dermal papilla cells play an important role in the proliferation/differentiation of hair follicle epithelial cells and the formation of hair, and it is considered that promoting the proliferation of follicle dermal papilla cells can prevent/ameliorate alopecia. Hitherto, for example, wild thyme extracts (see Patent Document 19) and the like are known as having follicle dermal papilla cell proliferation promotive actions.

There is a wide variety of causes and developmental mechanisms of inflammatory diseases, for example, contact dermatitis (rash), psoriasis, pemphigus vulgaris, atopic dermatitis, various other skin inflammatory diseases accompanied by rough skin, rheumatoid arthritis, osteoarthritis, asthma, etc. The causes are mainly known to be due to excessive production of nitric oxide (NO), release of histamine, increased activity of hyaluronidase, production of prostaglandin E₂ (PGE₂), etc.

Nitric oxide (NO) is a nitrogen oxide that causes air pollution, acid rain, etc., but in recent years, it has been found that the nitric oxide is a physiologically active substance that exhibits a variety of functions in vivo, such as a vascular endothelium-derived relaxation factor (EDRF), a neurotransmitter, and a factor that inhibits microorganisms/tumor cells in biological defense. In the biological defense, nitric oxide produced from macrophages particularly protects against bacterial and viral infections.

If large amounts of nitric oxide are biosynthesized, however, it will not be non-toxic to living organisms and will lead to destruction of self-tissues, causing pathological conditions such as worsening of inflammation, rheumatism, and diabetes. It is also known that large amounts of biosynthesized nitric oxide cause relaxation of vascular smooth muscle and excessive increase in permeability, leading to endotoxic shock due to a significant drop in blood pressure.

It is therefore important in inflammatory diseases to suppress excessive production of nitric oxide. Examples of known substances having an action of suppressing the production of nitric oxide include Angelica pubescens root, Aralia elata root bark, Dipsacus japonica Miq., Plantago seed, Enshi, madder root, Scutellaria barbata, sophora, and Sichuan pepper (see Non-Patent Document 14), extracts from plants belonging to the genus Hydrocotyle (see Patent Document 20), and maltulosyl arginine (see Patent Document 21).

Histamine release is a phenomenon in which histamine within mast cells is released outside the cells, and the released histamine causes an inflammatory response. Attempts have therefore been made to prevent or treat allergic diseases and inflammatory diseases using substances that inhibit or suppress histamine release. However, it is difficult to directly evaluate the release of histamine, and the release of histamine can be evaluated using the release of hexosaminidase, which has been confirmed to be released simultaneously with histamine release, as an indicator. Thus, by suppressing the release of hexosaminidase, the release of histamine can be simultaneously suppressed, and this is considered to be effective in preventing, treating, or ameliorating inflammatory diseases.

In addition, histamine is known to mediate information transmission between cells as a local transmitter, enhance the gastric acid secretion in the digestive organs, serve as a neurotransmitter in the central nervous system, and contribute to maintaining the arousal state. Here, excessive release of histamine causes ulcers in the digestive organs due to hyperacidity, and is a contributory factor to sleep disorders in the central nervous system. As described previously, by suppressing the release of hexosaminidase, the release of histamine can also be suppressed simultaneously, so it is considered that this can prevent, treat, or ameliorate gastric ulcers, sleep disorders, etc. caused by hyperacidity. For example, extracts from wisteria tea (see Patent Document 22) and the like are known as having hexosaminidase release suppressive actions.

Hyaluronidase is a hyaluronic acid hydrolyzing enzyme. Hyaluronate, which maintains the affinity for body tissues, is decomposed by ultraviolet rays, oxygen, etc. in a water-containing system, and as its molecular weight decreases, its water retention effect also decreases. Moreover, hyaluronic acid exists as intercellular tissue in vivo and is also involved in vascular permeability. Furthermore, hyaluronidase is present in mast cells, but is released by degranulation caused by its activation, and acts as an inflammatory chemical mediator. Therefore, inhibition of hyaluronidase activity is expected to enhance the moisture retention and prevent/alleviate inflammation. For example, extracts from plants of the genus Osbeckia (see Patent Document 23) and the like are known as having hyaluronidase activity inhibitory actions.

Inflammation is a complex reaction with symptoms such as reddening, edema, fever, pain, itching, and functional disorders. For example, when the skin is exposed to ultraviolet rays or comes into contact with irritating substances, inflammatory cytokines and the like are produced within the skin, causing skin inflammation. As a result, the skin tissue is damaged, causing various symptoms such as rough skin, reddening, edema, and pigmentation.

One example of inflammatory cytokines is prostaglandin E₂ (PGE₂). In the skin, PGE₂ is produced, for example, by keratinized cells (keratinocytes) or the like and causes skin inflammation. It has been revealed that cyclooxygenase-2 (COX-2), which is an inducible cyclooxygenase, is mainly involved in the production of prostaglandins during inflammation. Conceivable methods for treating or preventing skin inflammation therefore include suppressing the production of PGE₂ in keratinocytes or inhibiting the activity of COX-2. Pentaerythritol and the like are known as components having PGE₂ production suppressive actions on keratinocytes (see Patent Document 24).

The liver is an organ essential to life support that plays a central role in metabolism, and its main functions include metabolism of sugars/proteins/hormones, detoxification of harmful substances, bile production, and blood storage. The liver is an organ that does not show any symptoms even when it is damaged, but if the liver function is damaged due to alcohol drinking, over nutrition, drug abuse, hepatitis virus, etc., feeling of fatigue, feeling of malaise, loss of appetite, etc. will be exhibited, and yellow bile may appear. As liver dysfunction progresses, it may lead to lifestyle-related diseases such as hepatitis and cirrhosis, so improving the liver function and protecting it from disorders are extremely important for maintaining a healthy lifestyle.

Glutathione is known as an in vivo component that protects the liver. Glutathione is a tripeptide consisting of three amino acids: glutamic acid, cysteine, and glycine, and is a compound having cysteine residues, which are the major intracellular residues. It is known that glutathione is mainly produced in the liver and supplied throughout the body and protects the liver functions by being involved not only in scavenging radicals within cells, regulating cell functions through redox, and acting as an SH donor for various enzymes, but also in detoxification mechanisms especially in the liver. It is also known that glutathione in the liver is consumed in large quantities during the process of metabolizing ethanol when drinking alcohol, during the process of metabolizing and detoxifying drugs, or during other similar processes, and if the amount of glutathione in the liver decreases, it will cause liver dysfunctions such as acute or chronic alcoholic hepatitis and drug-induced hepatitis, and the amount of glutathione in the whole body will decrease, causing a variety of symptoms such as cataracts, Parkinson's disease, and the formation of age spots on the skin.

It is therefore considered that if the production of glutathione in the liver (hepatocytes) can be promoted, it will be expected that the liver functions are improved and various disorders caused by a decrease in glutathione can be prevented, treated, or ameliorated. Liquiritin (see the aforementioned Patent Document 11) and the like are known as having glutathione production promotive actions in hepatocytes.

Adenosine triphosphate (ATP) is known as a component that improves the liver functions. ATP is produced through the metabolism of glucose and fat and is used as an energy source. In the liver, ATP is used as an energy source for many chemical reactions carried out in the liver, such as the metabolism/detoxification of ethanol and drugs, and it is considered that if the production amount of ATP decreases, the efficiency of the metabolism/detoxification will decrease, and in addition to this, the energy required for activities such as work and exercise will be insufficient, resulting in fatigue.

It is therefore considered that if the ATP production in the liver can be promoted, it will be possible to prevent or ameliorate various symptoms caused by ethanol intake (e.g., hangovers, etc.), prevent, treat, or ameliorate the liver damage caused by drugs, and promote recovery from fatigue. Piper longum extracts (see Patent Document 25) and the like are known as having ATP production promotive actions in hepatocytes.

### PRIOR ART DOCUMENTS

### Patent Documents

[Patent Document 1] JP2006-056855A
[Patent Document 2] JP2004-083488A
[Patent Document 3] JP2002-201122A
[Patent Document 4] JP2003-146837A
[Patent Document 5] JP2005-022993A
[Patent Document 6] JP2003-300893A
[Patent Document 7] JP2010-090035A
[Patent Document 8] JP2006-063033A
[Patent Document 9] JP2006-056854A
[Patent Document 10] JP2003-321373A
[Patent Document 11] JP2009-256272A
[Patent Document 12] JP2009-269889A
[Patent Document 13] JP2007-099698A
[Patent Document 14] JP2009-191039A
[Patent Document 15] JP2012-219047A
[Patent Document 16] JP2009-256244A
[Patent Document 17] JP2013-023487A
[Patent Document 18] JP2010-184915A
[Patent Document 19] JP2006-219407A
[Patent Document 20] JP2006-28036A
[Patent Document 21] JP2010-90076A
[Patent Document 22] JP2003-012532A
[Patent Document 23] JP2003-055242A
[Patent Document 24] JP2015-214533A
[Patent Document 25] JP2011-184381A

### Non-Patent Documents

[Non-patent Document 1] “Folia Pharmacologica Japonica,” 2005, Vol. 125, No. 6, p. 379-384
[Non-patent document 2] J. Cell Biol., 1992, Vol. 119, No. 3, p. 695-703
[Non-patent Document 3] J. Invest. Dermatol., 1979, Vol. 73, No. 1, p. 59-66
[Non-patent Document 4] J. Dermatol., 1993, Vol. 20, No. 1, p. 1-6
[Non-patent Document 5] “Fragrance Journal,” 2004, Vol. 32, No. 11, p. 23-32
[Non-patent Document 6] Br. J. Dermatol., 2000, Vol. 143, Issue 3, p. 524-531
[Non-patent Document 7] “Fragrance Journal,” 2006, Vol. 34, No. 10, p. 19-23
[Non-patent Document 8] “Nat Genet.,” 2006, Vol. 38, No. 4, p. 441-446
[Non-patent Document 9] “Fragrance Journal Extra Edition,” 2000, Vol. 17, p. 14-19
[Non-patent Document 10] “Arch. Dermatol. Res.,” 1996, Vol. 288, p. 442-446
[Non-patent Document 11] J. Cell Biol., 2002, Vol. 156, pp. 1099-1111
[Non-patent Document 12] Journal of Japanese Cosmetic Science Society, 2007, Vol. 31, pp. 296-301
[Non-patent Document 13] Trends Genet., 1992, Vol. 8, Issue 2, p. 55-61
[Non-patent Document 14] “Journal of traditional medicines,” 1998, Vol. 15, p. 302-303

### SUMMARY OF THE INVENTION

### Problems to be Solved by the Invention

An object of the present invention is to find, from among compounds derived from natural products, those having excellent actions in an anti-metabolic syndrome action, a skin whitening action, an anti-aging action, a hair growth action, an anti-inflammatory action, or a liver function-improving action and provide an anti-metabolic syndrome agent, a skin whitening agent, an anti-aging agent, a hair growth agent, an anti-inflammatory agent, and a liver function-improving agent that contain the above compounds as the active ingredients.

Another object of the present invention is to provide an oral composition, a skin cosmetic, and a hair cosmetic that are compounded with compounds derived from natural products having excellent actions in an anti-metabolic syndrome action, a skin whitening action, an anti-aging action, a hair growth action, an anti-inflammatory action, or a liver function-improving action and that are suitable for an anti-metabolic syndrome application, a skin whitening application, an anti-aging application, a hair growth application, an anti-inflammatory application, and a liver function-improving application.

### Means for Solving the Problems

To solve the above problems, the anti-metabolic syndrome agent, skin whitening agent, anti-aging agent, hair growth agent, anti-inflammatory agent, and liver function-improving agent of the present invention are characterized by comprising one or more active ingredients selected from the group consisting of compounds 1 to 3 represented by General Formula (I) below.

Additionally or alternatively, the oral composition, skin cosmetic, and hair cosmetic of the present invention are characterized by compounding one or more selected from the group consisting of compounds 1 to 3 represented by General Formula (I) below.

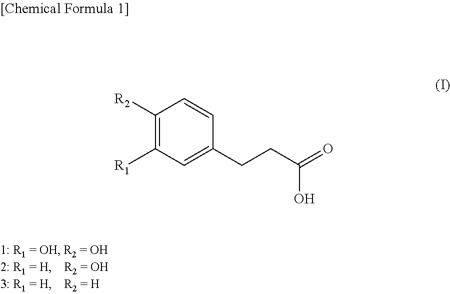

### Advantageous Effect of the Invention

According to the present invention, by using the one or more active ingredients selected from the group consisting of compounds 1 to 3 represented by the above General Formula (I), it is possible to provide the anti-metabolic syndrome agent, skin whitening agent, anti-aging agent, hair growth agent, anti-inflammatory agent, and liver function-improving agent that have excellent actions and effects.

Additionally or alternatively, according to the present invention, by compounding one or more selected from the group consisting of compounds 1 to 3 represented by the above General Formula (I), it is possible to provide the oral composition, skin cosmetic, and hair cosmetic that are suitable for an anti-metabolic syndrome application, a skin whitening application, an anti-aging application, a hair growth application, an anti-inflammatory application, or a liver function-improving application.

### EMBODIMENTS FOR CARRYING OUT THE INVENTION

One or more embodiments of the present invention will be described below.

### <Phenylpropionic Acids>

The anti-metabolic syndrome agent, skin whitening agent, anti-aging agent, hair growth agent, anti-inflammatory agent, and liver function-improving agent according to the present embodiment are characterized by comprising one or more active ingredients selected from the group consisting of compounds 1 to 3 represented by General Formula (I) below.

Additionally or alternatively, the oral composition, skin cosmetic, and hair cosmetic according to the present embodiment are characterized by compounding one or more selected from the group consisting of compounds 1 to 3 represented by General Formula (I) below.

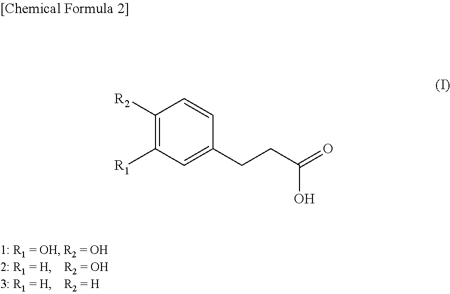

The compounds represented by the above General Formula (I) are all phenylpropionic acid derivatives, and the compound 1 is 3,4-dihydroxyhydrocinnamic acid. The compound 2 is 3-(4-hydroxyphenyl)propionic acid, and the compound 3 is 3-phenylpropionic acid.

In the present specification, one or more compounds represented by the above formula (I) may be referred to as “phenylpropionic acids,” hereinafter.

The above phenylpropionic acids can be produced, for example, by isolation/purification from plant extracts containing phenylpropionic acids. In this case, such plant extracts containing phenylpropionic acids can be obtained by a method that is commonly used for extraction from plants. Examples of plants containing the above phenylpropionic acids include rice, barley, wheat, soybeans, adzuki beans, and corn.

The above phenylpropionic acids can also be produced, for example, through fermenting 3,4-dihydroxycinnamic acid, 4-hydroxycinnamic acid, or cinnamic acid (these three types of compounds may be collectively referred to as “cinnamic acids,” hereinafter) or a composition containing them (e.g., a crushed material or extract of a plant, or the like) with a microorganism having phenolic acid reductase to convert cinnamic acids into phenylpropionic acids and then extracting/isolating/purifying the resulting fermented product. Examples of compositions containing cinnamic acids include crushed products and extracts of plants such as coffee, wheat, corn, tomato, mate, mugwort, and burdock. Cinnamic acids are constituents of lignin in woody plants and herbaceous plants, and lignin or a composition containing it may therefore be used as a fermentation raw material. On the other hand, examples of microorganisms having phenolic acid reductase include lactic acid bacteria such as Lactobacillus plantarum, Lactobacillus fermentum, Lactobacillus gasseri, Lactobacillus johnsonii, Lactobacillus crispatus, Lactobacillus acidophilus, Lactobacillus amylovorus, Lactobacillus delbrueckii, Lactobacillus buchneri, Lactobacillus kefiranofaciens, and Lactobacillus gallinarum and Enterococcus faecalis.

The method of extracting/isolating/purifying the above phenylpropionic acids from the above plants or fermented products is not particularly limited, and can be carried out according to an ordinary method. For example, the extraction process may be performed through drying the above plants or fermented products as extraction raw materials, then pulverizing them as they are or using a coarse pulverizer, and subjecting them to extraction with an extraction solvent. Drying may be performed in the sun or using a commonly used dryer. They may also be used as extraction raw materials after being subjected to preprocessing such as degreasing with a nonpolar solvent such as hexane. By performing preprocessing such as degreasing, the extraction process using a polar solvent can be performed efficiently.

It is preferred to use a polar solvent as the extraction solvent, and examples of the polar solvent include water, hydrophilic organic solvents, etc., which may be used alone or in combination of two or more at room temperature or a temperature equal to or lower than the boiling point of the solvent.

Examples of water that can be used as the extraction solvent include pure water, tap water, well water, mineral spring water, mineral water, hot spring water, spring water, fresh water, etc. and those that have been subjected to various processes. Examples of the processes applied to water include purification, heating, sterilization, filtration, ion exchange, osmotic pressure adjustment, and buffering. Therefore, examples of water that can be used as the extraction solvent in the present embodiment also include purified water, hot water, ion exchange water, physiological saline, phosphate buffer solution, and phosphate buffered saline.

Examples of hydrophilic organic solvents that can be used as extraction solvents include lower aliphatic alcohols whose carbon number is 1 to 5, such as methanol, ethanol, propyl alcohol, and isopropyl alcohol; polyhydric alcohols whose carbon number is 2 to 5, such as 1,3-butylene glycol, propylene glycol, and glycerin; and lower aliphatic ketones such as acetone and methyl ethyl ketone.

When a mixed solution of two or more polar solvents is used as the extraction solvent, the mixing ratio is arbitrary and can be adjusted as appropriate. For example, when a mixed solution of water and a hydrophilic organic solvent is used as the extraction solvent, they can be mixed and used at any ratio, that is, more than 0:100 and less than 100:0 (volume ratio, here and hereinafter), and the mixing ratio can be adjusted as appropriate.

For example, when a mixed solution of water and lower aliphatic alcohol is used as the extraction solvent, the mixing ratio (volume ratio) of water and lower aliphatic alcohol can be 9:1 or more in an embodiment or 7:3 or more in another embodiment, or the mixing ratio of water and lower aliphatic alcohol can be 1:9 or less in an embodiment or 2:8 or less in another embodiment. Additionally or alternatively, when a mixed solution of water and polyhydric alcohol is used, the mixing ratio of water and polyhydric alcohol can be 8:2 or more in an embodiment or 1:9 or less in another embodiment, and when a mixed solution of water and lower aliphatic ketone is used, the mixing ratio of water and lower aliphatic ketone can be 9:1 or more in an embodiment or 2:8 or less in another embodiment.

The extraction process is not particularly limited, provided that the soluble components contained in the extraction raw material can be eluted into the extraction solvent, and can be performed according to an ordinary method. For example, the extraction liquid can be obtained through immersing the extraction raw material in an extraction solvent of 5 to 15 times the amount of the extraction raw material (mass ratio), extracting the soluble components at an ordinary temperature or under reflux heating, and then removing the extraction residue by filtration. When the solvent is distilled off from the resulting extraction liquid, a paste-like concentrate is obtained, and when this concentrate is further dried, a dry product is obtained.

The method of isolating/purifying the above phenylpropionic acids from the extraction liquid obtained as above, the concentrate of the extraction liquid, or the dried product of the extraction liquid is not particularly limited, and can be carried out by an ordinary method. Examples of the method include a method of dissolving the extract in a developing solvent and subjecting it to column chromatography using a porous material such as silica gel or alumina, a porous resin such as styrene-divinylbenzene copolymer or polymethacrylate, or the like to collect a fraction containing the phenylpropionic acids. In this case, the developing solvent may be appropriately selected depending on the stationary phase used. For example, when extracts are separated by normal phase chromatography using silica gel as the stationary phase, examples of the developing solvent include chloroform:methanol=95:5. Furthermore, the fraction containing phenylpropionic acids obtained by column chromatography may be purified using any organic compound purification means such as reverse phase silica gel chromatography using ODS, recrystallization, liquid-liquid countercurrent extraction, or column chromatography using ion exchange resin.

### <Anti-Metabolic Syndrome Agent, Skin Whitening Agent, Anti-Aging Agent, Hair Growth Agent, Anti-Inflammatory Agent, and Liver Function-Improving Agent>

The above phenylpropionic acids obtained in the above manner have excellent actions in the anti-metabolic syndrome action, skin whitening action, anti-aging action, hair growth action, anti-inflammatory action, and liver function-improving action and can therefore be used as the active ingredients of an anti-metabolic syndrome agent, a skin whitening agent, an anti-aging agent, a hair growth agent, an anti-inflammatory agent, and a liver function-improving agent. Additionally or alternatively, the above phenylpropionic acids can be used for producing an anti-metabolic syndrome agent, a skin whitening agent, an anti-aging agent, a hair growth agent, an anti-inflammatory agent, or a liver function-improving agent.

The anti-metabolic syndrome agent, skin whitening agent, anti-aging agent, hair growth agent, anti-inflammatory agent, and liver function-improving agent of the present embodiment can be used in a wide range of applications such as pharmaceuticals, quasi-drugs, and cosmetics.

Here, the anti-metabolic syndrome action possessed by the above phenylpropionic acids is preferably exerted based on a cyclic AMP (cAMP) phosphodiesterase activity inhibitory action and/or a dipeptidyl peptidase IV (DPP IV) activity inhibitory action. However, the anti-metabolic syndrome action possessed by the above phenylpropionic acids is not limited to the anti-metabolic syndrome action exerted based on the above action or actions.

The above phenylpropionic acids can be used for a cAMP phosphodiesterase activity inhibitory application or a DPP IV activity inhibitory application, respectively, by using their cAMP phosphodiesterase activity inhibitory action or DPP IV activity inhibitory action. That is, the anti-metabolic syndrome agent of the present embodiment can also be used as a cAMP phosphodiesterase activity inhibitory agent or DPP IV activity inhibitory agent containing the above phenylpropionic acids as the active ingredients.

The skin whitening action possessed by the above phenylpropionic acids is preferably exerted based on a tyrosinase activity inhibitory action and/or a melanin production suppressive action. However, the skin whitening action possessed by the above phenylpropionic acids is not limited to the skin whitening action exerted based on the above action or actions.

The above phenylpropionic acids can be used for a tyrosinase activity inhibitory application or a melanin production suppressive application, respectively, by using their tyrosinase activity inhibitory action or melanin production suppressive action. That is, the skin whitening agent of the present embodiment can also be used as a tyrosinase activity inhibitory agent or melanin production suppressive agent containing the above phenylpropionic acids as the active ingredients.

The anti-aging action possessed by the above phenylpropionic acids is preferably exerted based on one or more actions selected from the group consisting of a type I collagen production promotive action, an elastin production promotive action, a hyaluronic acid production promotive action, an elastase activity inhibitory action, an HAS3 mRNA expression promotive action, a laminin-332 production promotive action, an epidermal keratinocyte proliferation promotive action, an ATP production promotive action, a glutathione production promotive action, a transglutaminase-1 (TGM1) mRNA expression promotive action, a serine palmitoyltransferase (SPT) mRNA expression promotive action, an aquaporin 3 (AQP3) mRNA expression promotive action, a filaggrin mRNA expression promotive action, a claudin-1 mRNA expression promotive action, a claudin-4 mRNA expression promotive action, an occludin mRNA expression promotive action, an advanced glycation end product (AGEs) formation suppressive action, and an advanced glycation end product (AGEs) decomposition promotive action. However, the anti-aging action possessed by the above phenylpropionic acids is not limited to the anti-aging action exerted based on the above action or actions.

The above phenylpropionic acids can be used for a type I collagen production promotion application, an elastin production promotion application, a hyaluronic acid production promotion application, an elastase activity inhibitory application, an HAS3 mRNA expression promotion application, a laminin-332 production promotion application, an epidermal keratinocyte proliferation promotion application, an ATP production promotion application, a glutathione production promotion application, a TGM1 mRNA expression promotion application, an SPT mRNA expression promotion application, an AQP3 mRNA expression promotion application, a filaggrin mRNA expression promotion application, a claudin-1 mRNA expression promotion application, a claudin-4 mRNA expression promotion application, an occludin mRNA expression promotion application, an AGEs formation suppressive application, or an AGEs decomposition promotion application, respectively, by using their type I collagen production promotive action, elastin production promotive action, hyaluronic acid production promotive action, elastase activity inhibitory action, HAS3 mRNA expression promotive action, laminin-332 production promotive action, epidermal keratinocyte proliferation promotive action, ATP production promotive action, glutathione production promotive action, TGM1 mRNA expression promotive action, SPT mRNA expression promotive action, AQP3 mRNA expression promotive action, filaggrin mRNA expression promotive action, claudin-1 mRNA expression promotive action, claudin-4 mRNA expression promotive action, occludin mRNA expression promotive action, AGEs formation suppressive action, or AGEs decomposition promotive action.

That is, the anti-aging agent of the present embodiment can also be used as a type I collagen production promotive agent, elastin production promotive agent, hyaluronic acid production promotive agent, elastase activity inhibitory agent, HAS3 mRNA expression promotive agent, laminin-332 production promotive agent, epidermal keratinocyte proliferation promotive agent, ATP production promotive agent, glutathione production promotive agent, TGM1 mRNA expression promotive agent, SPT mRNA expression promotive agent, AQP3 mRNA expression promotive agent, filaggrin mRNA expression promotive agent, claudin-1 mRNA expression promotive agent, claudin-4 mRNA expression promotive agent, occludin mRNA expression promotive agent, AGEs formation suppressive agent, or AGEs decomposition promotive agent containing the above phenylpropionic acids as the active ingredients.

The hair growth action possessed by the above phenylpropionic acids is preferably exerted based on a testosterone 5α-reductase activity inhibitory action and/or a follicle dermal papilla cell proliferation promotive action. However, the hair growth action possessed by the above phenylpropionic acids is not limited to the hair growth action exerted based on the above action or actions.

The above phenylpropionic acids can be used for a testosterone 5α-reductase activity inhibitory application or a follicle dermal papilla cell proliferation promotive application, respectively, by using their testosterone 5α-reductase activity inhibitory action or follicle dermal papilla cell proliferation promotive action. That is, the hair growth agent of the present embodiment can also be used as a testosterone 5α-reductase activity inhibitory agent or follicle dermal papilla cell proliferation promotive agent containing the above phenylpropionic acids as the active ingredients.

The anti-inflammatory action possessed by the above phenylpropionic acids is preferably exerted based on one or more actions selected from the group consisting of a nitric oxide (NO) production suppressive action, a hyaluronidase activity inhibitory action, a hexosaminidase release suppressive action, and a prostaglandin E₂ (PGE₂) activity inhibitory action. However, the anti-inflammatory action possessed by the above phenylpropionic acids is not limited to the anti-inflammatory action exerted based on the above action or actions.

The above phenylpropionic acids can be used for an NO production suppressive application, a hyaluronidase activity inhibitory application, a hexosaminidase release suppressive application, or a PGE₂ production promotive application, respectively, by using their NO production suppressive action, hyaluronidase activity inhibitory action, hexosaminidase release suppressive action, or PGE₂ production promotive action. That is, the anti-inflammatory agent of the present embodiment can also be used as an NO production suppressive agent, hyaluronidase activity inhibitory agent, hexosaminidase release suppressive agent, or PGE₂ production promotive action

The liver function-improving action possessed by the above phenylpropionic acids is preferably exerted based on a hepatocyte glutathione production promotive action and/or a hepatocyte adenosine triphosphate (ATP) production promotive action. However, the liver function-improving action possessed by the above phenylpropionic acids is not limited to the liver function-improving action exerted based on the above action or actions.

The above phenylpropionic acids can be used for a hepatocyte glutathione production promotive application or a hepatocyte ATP production promotive application, respectively, by using their hepatocyte glutathione production promotive action or hepatocyte ATP production promotive action. That is, the liver function-improving agent of the present embodiment can also be used as a hepatocyte glutathione production promotive agent or a hepatocyte ATP production promotive agent containing the above phenylpropionic acids as the active ingredients.

Compositions containing phenylpropionic acids as substitute for the above phenylpropionic acids isolated may be used as the active ingredients of the anti-metabolic syndrome agent, skin whitening agent, anti-aging agent, hair growth agent, anti-inflammatory agent, or liver function-improving agent according to the present embodiment. Here, the “compositions containing phenylpropionic acids” in the present embodiment include extracts obtained from plants containing the above phenylpropionic acids as the extraction raw materials, fermented products containing phenylpropionic acids, and extracts obtained using the fermented products as the extraction raw materials. The “extracts” include an extraction liquid obtained by an extraction process, a diluted or concentrated liquid of the extraction liquid, or a dried product obtained by drying the extraction liquid.

When the compositions containing the above phenylpropionic acids are used as the active ingredients of the anti-metabolic syndrome agent, skin whitening agent, anti-aging agent, hair growth agent, anti-inflammatory agent, or liver function-improving agent according to the present embodiment, the content of phenylpropionic acids in the compositions is preferably 0.1 mass % or more, more preferably 5 mass % or more, and particularly preferably 50 mass % or more. By using the phenylpropionic acids with increased purity as the active ingredients, the anti-metabolic syndrome agent, skin whitening agent, anti-aging agent, hair growth agent, anti-inflammatory agent, or liver function-improving agent can be obtained with even more excellent actions and effects.

The anti-metabolic syndrome agent, skin whitening agent, anti-aging agent, hair growth agent, anti-inflammatory agent, or liver function-improving agent according to the present embodiment may consist only of the above phenylpropionic acids or compositions containing phenylpropionic acids or may also be obtaining by formulating phenylpropionic acids or compositions containing phenylpropionic acids.

The anti-metabolic syndrome agent, skin whitening agent, anti-aging agent, hair growth agent, anti-inflammatory agent, or liver function-improving agent according to the present embodiment can be formulated into any dosage form such as powder, granules, tablets, or liquid according to an ordinary method using a pharmaceutically acceptable carrier such as dextrin or cyclodextrin or any other auxiliary agent. In this case, examples of usable auxiliary agents include an excipient, a binder, a disintegrant, a lubricant, a stabilizer, and a flavoring/odor ameliorating agent. The anti-metabolic syndrome agent, skin whitening agent, anti-aging agent, hair growth agent, anti-inflammatory agent, and liver function-improving agent can be used by being compounded with other compositions (e.g., oral compositions, skin cosmetics, etc. described later) or can also be used as liquids for external use, adhesive skin patches, etc.

When the anti-metabolic syndrome agent, skin whitening agent, anti-aging agent, hair growth agent, anti-inflammatory agent, or liver function-improving agent according to the present embodiment is formulated, the content of the above phenylpropionic acids or compositions containing phenylpropionic acids is not particularly limited and can be set as appropriate depending on the purpose.

The anti-metabolic syndrome agent, skin whitening agent, anti-aging agent, hair growth agent, anti-inflammatory agent, or liver function-improving agent according to the present embodiment can be used as active ingredients by compounding other natural extracts or the like having an anti-metabolic syndrome action, skin whitening action, anti-aging action, hair growth action, anti-inflammatory action, or liver function-improving action as necessary together with the above phenylpropionic acids or compositions containing phenylpropionic acids.

Examples of methods for administering the anti-metabolic syndrome agent, skin whitening agent, anti-aging agent, hair growth agent, anti-inflammatory agent, or liver function-improving agent according to the present embodiment include oral administration, transdermal administration, intraperitoneal administration, intravenous administration, and subcutaneous administration. Depending on the type of disease, a method suitable for its prevention or treatment may be appropriately selected. The dosage amount of the anti-metabolic syndrome agent, skin whitening agent, anti-aging agent, hair growth agent, anti-inflammatory agent, or liver function-improving agent according to the present embodiment may be increased or decreased as appropriate depending on the type of disease, severity, individual differences between patients, administration method, administration period, etc.

The anti-metabolic syndrome agent of the present embodiment can promote fat decomposition through the anti-metabolic syndrome action, preferably through the cAMP phosphodiesterase activity inhibitory action, and as a result, it is possible to prevent/ameliorate adiposity and various lifestyle-related diseases associated with the adiposity, such as arteriosclerosis, diabetes, and metabolic syndrome. The anti-metabolic syndrome agent of the present embodiment can also prevent/ameliorate type 2 diabetes, obesity, hypertension, insulin resistance, etc. through the anti-metabolic syndrome action, preferably through the DPP IV activity inhibitory action. Fortunately, however, the anti-metabolic syndrome agent of the present embodiment can be used not only for these applications but also for all applications that are meaningful in exerting the anti-metabolic syndrome action, preferably the cAMP phosphodiesterase activity inhibitory action or DPP IV activity inhibitory action.

For example, the anti-metabolic syndrome agent of the present embodiment or the aforementioned cAMP phosphodiesterase activity inhibitory agent suppresses the decomposition of cAMP and can therefore suppress platelet aggregation, thereby preventing, treating, or ameliorating allergic diseases and various inflammatory diseases.

Moreover, the anti-metabolic syndrome agent of the present embodiment or the aforementioned DPP IV activity inhibitory agent can prevent/treat autoimmune diseases such as rheumatoid arthritis and transplantation rejection through the DPP IV activity inhibitory action, and can also prevent/treat neurological disorders such as algia, neurodegenerative diseases, and neuropsychiatric diseases (e.g. sciatica, Alzheimer's disease, depression, etc.); growth hormone deficiency and diseases for which growth hormone is used; cancer (e.g., T-cell lymphoma, acute lymphoblastic leukemia, thyroid cancer, basal cell carcinoma, breast cancer, etc.); HIV infection (AIDS), etc.

The skin whitening agent of the present embodiment can prevent/ameliorate pigmentation such as skin darkening, age spots, or freckles through the skin whitening action, preferably through the tyrosinase activity inhibitory action and/or the melanin production suppressive action. Fortunately, however, the skin whitening agent of the present embodiment can be used not only for these applications but also for all applications that are meaningful in exerting the skin whitening action, preferably the tyrosinase activity inhibitory action or melanin production suppressive action.

The anti-aging agent of the present embodiment can prevent, treat, or ameliorate skin aging symptoms such as the formation of skin wrinkles, loss of elasticity, and loss of moisturizing function through the anti-aging action, preferably through one or more actions selected from the group consisting of a type I collagen production promotive action, an elastin production promotive action, a hyaluronic acid production promotive action, an elastase activity inhibitory action, an HAS3 mRNA expression promotive action, a laminin-332 production promotive action, an epidermal keratinocyte proliferation promotive action, an ATP production promotive action, a glutathione production promotive action, a TGM1 mRNA expression promotive action, an SPT mRNA expression promotive action, an AQP3 mRNA expression promotive action, a filaggrin mRNA expression promotive action, a claudin-1 mRNA expression promotive action, a claudin-4 mRNA expression promotive action, an occludin mRNA expression promotive action, an AGEs formation suppressive action, and an AGEs decomposition promotive action.

Fortunately, however, the anti-aging agent of the present embodiment can be used not only for these applications but also for all applications that are meaningful in exerting the anti-aging action, preferably the type I collagen production promotive action, elastin production promotive action, hyaluronic acid production promotive action, elastase activity inhibitory action, HAS3 mRNA expression promotive action, laminin-332 production promotive action, epidermal keratinocyte proliferation promotive action, ATP production promotive action, glutathione production promotive action, TGM1 mRNA expression promotive action, SPT mRNA expression promotive action, AQP3 mRNA expression promotive action, filaggrin mRNA expression promotive action, claudin-1 mRNA expression promotive action, claudin-4 mRNA expression promotive action, occludin mRNA expression promotive action, AGEs formation suppressive action, or AGEs decomposition promotive action.

For example, the anti-aging agent of the present embodiment or the aforementioned type I collagen production promotive agent can be used for applications such as: prevention, treatment, or amelioration of diseases caused by decreased collagen production, such as osteoporosis; promotion of regeneration of damaged tendons and ligaments; and promotion of healing of wounds or burns through the type I collagen production promotive action. Moreover, the anti-aging agent of the present embodiment or the aforementioned elastin production promotive agent or elastase activity inhibitory agent can be used for applications for prevention, treatment, or amelioration of pulmonary diseases such as emphysema and vascular diseases such as high blood pressure and aneurysms through the elastin production promotive action or elastase activity inhibitory action. Furthermore, the anti-aging agent of the present embodiment or the aforementioned hyaluronic acid production promotive agent can be used for applications such as: prevention, treatment, or amelioration of arthritis such as chronic rheumatoid arthritis, arthritis deformans, suppurative arthritis, gouty arthritis, traumatic arthritis, or osteoarthritis; and promotion of healing of wounds or burns through the hyaluronic acid production promotive action.

In addition to the aforementioned applications, the anti-aging agent of the present embodiment or the aforementioned laminin-332 production promotive agent can induce reconstruction of the basement membrane structure through the laminin-332 production promotive action, thereby treating/ameliorating wounds in the skin. Moreover, the anti-aging agent of the present embodiment or the aforementioned laminin-332 production promotive agent can be used as a prophylactic or therapeutic agent for diseases (such as epidermolysis bullosa) caused by laminin-332 deficiency (deletion).

In addition to the aforementioned applications, the anti-aging agent of the present embodiment or the aforementioned epidermal keratinocyte proliferation promotive agent restores the skin metabolism through the epidermal keratinocyte proliferation promotive action, and can be used for applications such as prevention, treatment, or amelioration of wrinkles, dullness, pigmentation, etc. and regenerative medicine. Moreover, the anti-aging agent of the present embodiment or the aforementioned ATP production promotive agent can promote the turnover through the ATP production promotive action to prevent/ameliorate the skin aging symptoms such as wrinkles, loss of texture, and decreased elasticity in the skin, and can restore the metabolic function of the skin, such as the shedding of corneocytes with abnormal accumulation of melanin from the stratum corneum, thereby preventing/ameliorating the symptoms such as dullness and pigmentation in the skin. Furthermore, the anti-aging agent of the present embodiment or the aforementioned ATP production promotive agent or glutathione production promotive agent can also be used in applications based on the hepatocyte ATP production promotive action or hepatocyte glutathione production promotive action, which will be described later.

In addition to the aforementioned applications, the anti-aging agent of the present embodiment or the aforementioned TGM1 mRNA expression promotive agent, SPT mRNA expression promotive agent, or filaggrin mRNA expression promotive agent can be used to strengthen the barrier function of the skin through the TGM1 mRNA expression promotive action, SPT mRNA expression promotive action, or filaggrin mRNA expression promotive action and can prevent, treat, or ameliorate the rough skin, dry skin, and other dry skin diseases (e.g., atopic dermatitis, psoriasis, ichthyosis, and the like). Moreover, the anti-aging agent of the present embodiment or the aforementioned filaggrin mRNA expression promotive agent or AQP3 mRNA expression promotive agent can ameliorate the moisture retention function, barrier function, etc. depending on the aging through the filaggrin mRNA expression promotive action or AQP3 mRNA expression promotive action.

In addition to the aforementioned applications, the anti-aging agent of the present embodiment or the aforementioned claudin-1 mRNA expression promotive agent, claudin-4 mRNA expression promotive agent, or occludin mRNA expression promotive agent can be used to promote the formation of tight junctions in the epidermal keratinocytes through the claudin-1 mRNA expression promotive action, the claudin-4 mRNA expression promotive action, or the occludin mRNA expression promotive action, and can thereby enhance the barrier function and moisture retention function of the skin and prevent or ameliorate skin symptoms such as dry skin, rough skin, atopic dermatitis, and various infections. Moreover, the anti-aging agent of the present embodiment or the aforementioned claudin-1 mRNA expression promotive agent, claudin-4 mRNA expression promotive agent, or occludin mRNA expression promotive agent can ameliorate the barrier function in the gastrointestinal tract and prevent or ameliorate inflammatory bowel diseases, food allergies, various infections transmitted from the gastrointestinal tract, etc.

In addition to the aforementioned applications, the anti-aging agent of the present embodiment or the aforementioned AGEs formation suppressive agent or AGEs decomposition promotive agent can be used to prevent or treat diabetic complications such as diabetic neuropathy, diabetic retinopathy, and diabetic nephropathy; arteriosclerosis caused by protein glycation reactions; osteoporosis and osteoarthritis caused by protein glycation reactions; etc. through the AGEs formation suppressive action or AGEs decomposition promotive action. Moreover, the anti-aging agent of the present embodiment or the aforementioned AGEs formation suppressive agent can prevent or suppress hair damage caused by protein glycation reactions through the AGEs formation suppressive action and thereby prevent or suppress hair stiffness, and can restore the elasticity and suppleness of the hair and add firmness/body to the hair.

The hair growth agent of the present embodiment can prevent, treat, or ameliorate alopecia or the like, such as male pattern alopecia, alopecia areata, or trichotillomania, through the hair growth action, preferably the testosterone 5α-reductase activity inhibitory action and/or the follicle dermal papilla cell proliferation promotive action, and may be particularly suitable for the prevention, treatment, or amelioration of the male pattern alopecia. Fortunately, however, the hair growth agent of the present embodiment can be used not only for these applications but also for all applications that are meaningful in exerting the hair growth action, preferably the testosterone 5α-reductase activity inhibitory action or the follicle dermal papilla cell proliferation promotive action.

For example, the hair growth agent of the present embodiment or the aforementioned testosterone 5α-reductase activity inhibitory agent can prevent, treat, or ameliorate diseases related to male hormones, for example, hirsutism, seborrhea, acne (such as comedo), benign prostatic hypertrophy, prostate tumors, premature male puberty, etc., through the testosterone 5α-reductase activity inhibitory action. Moreover, the hair growth agent of the present embodiment or the aforementioned follicle dermal papilla cell proliferation promotive agent can also be used for applications in the field of regenerative medicine, such as hair regeneration using follicle dermal papilla cells, through the follicle dermal papilla cell proliferation promotive action.

The anti-inflammatory agent of the present embodiment can prevent, treat, or ameliorate contact dermatitis (rash), psoriasis, pemphigus vulgaris, atopic dermatitis, and various other skin inflammatory diseases accompanied by rough skin through the anti-inflammatory action, preferably one or more actions selected from the group consisting of the NO production suppressive action, hyaluronidase activity inhibitory action, hexosaminidase release suppressive action, and PGE₂ production promotive action. Fortunately, however, the anti-inflammatory agent of the present embodiment can be used not only for these applications but also for all applications that are meaningful in exerting the anti-inflammatory action, preferably the NO production suppressive action, hyaluronidase activity inhibitory action, hexosaminidase release suppressive action, or PGE₂ production promotive action.

For example, the anti-inflammatory agent of the present embodiment or the aforementioned NO production suppressive agent, hyaluronidase activity inhibitory agent, hexosaminidase release suppressive agent, or PGE₂ production promotive agent can prevent, treat, or ameliorate rheumatoid arthritis, osteoarthritis, asthma, etc. through the NO production suppressive action, hyaluronidase activity inhibitory action, hexosaminidase release suppressive action, or PGE₂ production promotive action. Moreover, the anti-inflammatory agent of the present embodiment or the aforementioned hexosaminidase release suppressive agent can prevent, treat, or ameliorate gastric ulcers, sleep disorders, etc. caused by hyperacidity.

The liver function-improving agent of the present embodiment can improve the liver function through the liver function-improving action, preferably through the hepatocyte glutathione production promotive action and/or the hepatocyte ATP production promotive action. Examples of applications for improving or ameliorating the liver function include: prevention, treatment, or amelioration of symptoms caused by ethanol intake (e.g., hangovers, etc.); promotion of metabolism/decomposition of sugars, fats, etc.; prevention, treatment, or amelioration of fatty liver, hepatitis such as alcoholic hepatitis or drug-induced hepatitis, liver cirrhosis, etc.

Fortunately, however, the liver function-improving agent of the present embodiment can be used not only for these applications but also for all applications that are meaningful in exerting the liver function-improving action, preferably the hepatocyte glutathione production promotive action or the hepatocyte ATP production promotive action.

For example, the liver function-improving agent of the present embodiment or the aforementioned hepatocyte glutathione production promotive agent can be used for applications such as: prevention, treatment, or amelioration of diseases associated with a decrease in the in vivo glutathione concentration, such as cataracts and Parkinson's disease; prevention, treatment, or amelioration of pigmentation such as skin darkening, age spots, or freckles; etc. through the hepatocyte glutathione production promotive action. Moreover, the liver function-improving agent of the present embodiment or the aforementioned hepatocyte ATP production promotive agent can be used for applications such as recovery from feeling of fatigue/malaise, etc. through the hepatocyte ATP production promotive action.

The anti-metabolic syndrome agent, skin whitening agent, anti-aging agent, hair growth agent, anti-inflammatory agent, and liver function-improving agent of the present embodiment have excellent actions in the anti-metabolic syndrome action, skin whitening action, anti-aging action, hair growth action, anti-inflammatory action, and liver function-improving action, respectively, and can therefore be suitably used as reagents for research on the mechanisms of these actions.

### <Oral Composition>

The above phenylpropionic acids have excellent actions in the anti-metabolic syndrome action, skin whitening action, anti-aging action, hair growth action, anti-inflammatory action, and liver function-improving action, and are therefore suitable for being compounded in oral compositions. In this case, the above phenylpropionic acids or compositions containing phenylpropionic acids may be compounded as they are, or the anti-metabolic syndrome agent, skin whitening agent, anti-aging agent, hair growth agent, anti-inflammatory agent, or liver function-improving agent formulated from the phenylpropionic acids may also be compounded.

By compounding the oral composition with the above phenylpropionic acids or compositions containing phenylpropionic acids or with the anti-metabolic syndrome agent, skin whitening agent, anti-aging agent, hair growth agent, anti-inflammatory agent, or liver function-improving agent formulated from the phenylpropionic acids or compositions containing phenylpropionic acids, the oral composition can be suitable for the anti-metabolic syndrome application, skin whitening application, anti-aging application, hair growth application, anti-inflammatory application, or liver function-improving application. Among these, the anti-metabolic syndrome action and the liver function-improving action are suitable because they are likely to exhibit their actions and effects when given to the oral composition.

Here, the oral composition refers to a composition that has little risk of harming human health and is ingested orally or through gastrointestinal administration in normal social life, and is not restricted by administrative classifications such as foods, pharmaceuticals, and quasi-drugs. Therefore, the “oral composition” in the present embodiment refers to general foods, feeds, health foods, foods with health claims (foods for specified health uses, foods with nutrient function claims, foods and drinks with functional claims), quasi-drugs, pharmaceuticals, etc. that are ingested orally, and includes a wide variety of products. The oral composition according to the present embodiment is preferably an oral composition that can display the favorable actions possessed by the above phenylpropionic acids on the oral composition or its packaging, and particularly preferably any of foods with health claims (foods for specified health uses, foods with nutrient function claims, foods and drinks with functional claims), quasi-drugs, or pharmaceuticals.

When compounding the oral composition with the above phenylpropionic acids or compositions containing phenylpropionic acids or with the anti-metabolic syndrome agent, skin whitening agent, anti-aging agent, hair growth agent, anti-inflammatory agent, or liver function-improving agent formulated from the phenylpropionic acids or compositions containing phenylpropionic acids, the compounding amount of active ingredients therein can be changed as appropriate in consideration of the purpose of use, symptoms, gender, etc., but considering the daily intake of phenylpropionic acids as the additives, the daily intake of phenylpropionic acids for adults is preferably about 1 to 1000 mg. When the oral composition to be added is in the form of granules, tablets, or capsules, the compounding amount of the above phenylpropionic acids or compositions containing phenylpropionic acids or the anti-metabolic syndrome agent, skin whitening agent, anti-aging agent, hair growth agent, anti-inflammatory agent, or liver function-improving agent formulated from the phenylpropionic acids or compositions containing phenylpropionic acids is usually 0.1 to 100 mass % and preferably 5 to 100 mass % with respect to the oral composition to be added.

The oral composition of the present embodiment may be one in which the above phenylpropionic acids are compounded into any oral composition that does not interfere with the activity of the phenylpropionic acids, or may also be a nutritional supplement containing the above phenylpropionic acids as the main components.

When producing the oral composition of the present embodiment, the oral composition can be formed in any shape by adding arbitrary auxiliary agents, for example, sugars such as dextrin and starch; proteins such as gelatin, soybean protein, and corn protein; amino acids such as alanine, glutamine, and isoleucine; polysaccharides such as cellulose and gum arabic; oils and fats such as soybean oil and medium-chain fatty acid triglyceride; and the like.

Oral compositions that can be compounded with the above phenylpropionic acids are not particularly limited, but specific examples include beverages such as soft drinks, carbonated drinks, nutritional drinks, fruit drinks, and lactic acid drinks (including concentrated undiluted solutions of these drinks and powders for adjustment); frozen desserts such as ice cream, ice sherbet, and shaved ice; noodles such as buckwheat noodle, wheat noodle, bean thread noodle, dumpling skin, Chinese dumpling skin, Chinese noodle, and instant noodle; sweets such as lollipop, chewing gum, candy, gum, chocolate, compressed tablet candy, snacks, biscuits, jellies, jams, creams, and baked confectionery; fishery/livestock processed foods such as fish minced and steamed, ham, and sausage; dairy products such as processed milk and fermented milk; fats and oils and their processed foods such as salad oil, fritter oil, margarine, mayonnaise, shortening, whipped cream, and dressings; seasonings such as sauces and mop sauces; soups, stews, salads, side dishes, and pickles; and various other forms of health/nutritional supplements; tablets, capsules, drinks, etc. When the above phenylpropionic acids are compounded in these oral compositions, commonly used auxiliary raw materials and additives can be used in combination.

### <Skin Cosmetics and Hair Cosmetics>

The above phenylpropionic acids have excellent actions in the anti-metabolic syndrome action, skin whitening action, anti-aging action, hair growth action, anti-inflammatory action, and liver function-improving action and are therefore suitable for being compounded in skin cosmetics or hair cosmetics. In this case, the above phenylpropionic acids may be compounded without any modification, or the anti-metabolic syndrome agent, skin whitening agent, anti-aging agent, hair growth agent, anti-inflammatory agent, or liver function-improving agent formulated from the above phenylpropionic acids may be compounded.

By compounding the above phenylpropionic acids or the above anti-metabolic syndrome agent, skin whitening agent, anti-aging agent, hair growth agent, anti-inflammatory agent, or liver function-improving agent, the skin cosmetic or the hair cosmetic can be imparted with the anti-metabolic syndrome action, skin whitening action, anti-aging action, hair growth action, anti-inflammatory action, or liver function-improving action, and can be used for the anti-metabolic syndrome application, skin whitening application, anti-aging application, hair growth application, anti-inflammatory application, or liver function-improving application.

Among these, the skin whitening action, anti-aging action, and anti-inflammatory action and their effects are likely to be exhibited when the agents or the like are compounded in skin cosmetics. That is, the skin cosmetics are particularly suitable for the skin whitening application, anti-aging application, or anti-inflammatory application. Moreover, the hair growth action, anti-aging action, and anti-inflammatory action and their effects are likely to be exhibited when the agents or the like are compounded in hair cosmetics. That is, the hair cosmetics are particularly suitable for the hair growth application, anti-aging application, or anti-inflammatory application.

There are no particular restrictions on the types of skin cosmetics or hair cosmetics that can be compounded with the above phenylpropionic acids or the above anti-metabolic syndrome agent, skin whitening agent, anti-aging agent, hair growth agent, anti-inflammatory agent, or liver function-improving agent. Examples of the skin cosmetics include ointments, creams, milky lotions, beauty wash, lotions, gels, beauty oils, packs, and foundations. Examples of the hair cosmetics include hair tonics, hair creams, hair liquids, shampoos, pomades, and hair conditioners.

When the skin cosmetics or hair cosmetics are compounded with the above phenylpropionic acids or the above anti-metabolic syndrome agent, skin whitening agent, anti-aging agent, hair growth agent, anti-inflammatory agent, or liver function-improving agent, the compounding amount can be adjusted as appropriate depending on the types of skin cosmetics or hair cosmetics. The suitable compounding ratio is about 0.0001 to 10 mass %, and a particularly suitable compounding ratio is about 0.001 to 1 mass %, calculated as a standard extract.

The skin cosmetics or hair cosmetics of the present embodiment can be used in combination with main agents, auxiliary agents, or other components used in the production of usual skin cosmetics or hair cosmetics, for example, astringents, bactericidal/antibacterial agents, skin whitening agents, ultraviolet absorbers, humectants, cell activators, anti-inflammatory/anti-allergic agents, antioxidant/active oxygen scavengers, oils and fats, waxes, hydrocarbons, fatty acids, alcohols, esters, surfactants, fragrances, etc., provided that they do not interfere with the anti-metabolic syndrome action, skin whitening action, anti-aging action, hair-growth action, anti-inflammatory action, or liver function-improving action possessed by the phenylpropionic acids. Such combination use allows the product to be more general, and the synergistic actions with other active ingredients used in combination may provide better effects than would normally be expected.

The skin cosmetic of the present embodiment can achieve the following effects: prevention, treatment, or amelioration of pigmentation such as skin darkening, spots, and freckles; prevention, treatment, or amelioration of skin aging symptoms such as the formation of skin wrinkles, loss of elasticity, and loss of moisturizing function; promotion of healing of wounds or burns; prevention, treatment, or amelioration of the rough skin, dry skin, and other dry skin diseases (e.g., atopic dermatitis, psoriasis, ichthyosis, and the like); prevention, treatment, or amelioration of diseases related to male hormones, such as seborrhea and acne (such as comedo); prevention, treatment, or amelioration of contact dermatitis (rash), psoriasis, pemphigus vulgaris, and various other skin inflammatory diseases accompanied by rough skin; prevention, treatment, or amelioration of adiposity and lifestyle-related diseases associated with the adiposity, such as arteriosclerosis, diabetes, and metabolic syndrome; etc. through one or more actions possessed by the above phenylpropionic acids selected from the group consisting of: the skin whitening action, tyrosinase activity inhibitory action, and melanin production suppressive action; the anti-aging action, type I collagen production promotive action, elastin production promotive action, hyaluronic acid production promotive action, elastase activity inhibitory action, HAS3 mRNA expression promotive action, laminin-332 production promotive action, epidermal keratinocyte proliferation promotive action, ATP production promotive action, glutathione production promotive action, TGM1 mRNA expression promotive action, SPT mRNA expression promotive action, AQP3 mRNA expression promotive action, filaggrin mRNA expression promotive action, claudin-1 mRNA expression promotive action, claudin-4 mRNA expression promotive action, occludin mRNA expression promotive action, AGEs formation suppressive action, and AGEs decomposition promotive action; the hair growth action, testosterone 5α-reductase activity inhibitory action, and follicle dermal papilla cell proliferation promotive action; the anti-inflammatory action, NO production suppressive action, hyaluronidase activity inhibitory action, hexosaminidase release suppressive action, and PGE₂ production promotive action; the anti-metabolic syndrome action, cAMP phosphodiesterase activity inhibitory action, and DPP IV activity inhibitory action; and the liver function-improving action, hepatocyte glutathione production promotive action, and hepatocyte ATP production promotive action.

The hair cosmetic of the present embodiment can achieve the following effects: prevention, treatment, or amelioration of alopecia such as male pattern alopecia, alopecia areata, or trichotillomania; prevention, treatment, or amelioration of contact dermatitis (rash), psoriasis, pemphigus vulgaris, and various other skin inflammatory diseases accompanied by rough skin; treatment of the rough skin, dry skin, and other dry skin diseases (e.g., atopic dermatitis, psoriasis, ichthyosis, and the like); etc. through one or more actions possessed by the above phenylpropionic acids selected from the group consisting of: the hair growth action, testosterone 5α-reductase activity inhibitory action, and follicle dermal papilla cell proliferation promotive action; the anti-inflammatory action, NO production suppressive action, hyaluronidase activity inhibitory action, hexosaminidase release suppressive action, and PGE₂ production promotive action; the anti-aging action, type I collagen production promotive action, elastin production promotive action, hyaluronic acid production promotive action, elastase activity inhibitory action, HAS3 mRNA expression promotive action, laminin-332 production promotive action, epidermal keratinocyte proliferation promotive action, ATP production promotive action, glutathione production promotive action, TGM1 mRNA expression promotive action, SPT mRNA expression promotive action, AQP3 mRNA expression promotive action, filaggrin mRNA expression promotive action, claudin-1 mRNA expression promotive action, claudin-4 mRNA expression promotive action, occludin mRNA expression promotive action, AGEs formation suppressive action, and AGEs decomposition promotive action; the anti-metabolic syndrome action, cAMP phosphodiesterase activity inhibitory action, and DPP IV activity inhibitory action; the skin whitening action, tyrosinase activity inhibitory action, and melanin production suppressive action; and the liver function-improving action, hepatocyte glutathione production promotive action, and hepatocyte ATP production promotive action.

That the anti-metabolic syndrome agent, skin whitening agent, anti-aging agent, hair growth agent, anti-inflammatory agent, liver function-improving agent, oral composition, skin cosmetic, and hair cosmetic of the present embodiment are suitably applied to humans, but can also be applied to animals other than humans (e.g., mice, rats, hamsters, dogs, cats, cows, pigs, monkeys, etc.), provided that the respective actions and effects are achieved.

### EXAMPLES

Hereinafter, the present invention will be specifically described with reference to testing examples, but the present invention is not limited to the following examples. In these testing examples, the following commercially available compounds were used as test samples.

TABLE 1

| | | | Sample |
|---|---|---|---|
| | Name of compound | Manufacture | No.. |
| Compound 1 | 3,4-Dihydroxyhydrocinnamic | Sigma-Aldrich | Sam- |
| | acid | | ple 1 |
| Compound 2 | 3-(4-Hydroxyphenyl)propionic | Sigma-Aldrich | Sam- |
| | acid | | ple 2 |
| Compound 3 | 3-phenylpropionic acid | Tokyo | Sam- |
| | | Chemical | ple 3 |
| | | Industry Co., | |
| | | Ltd. (TCI) | |

### <Testing Example 1> Cyclic AMP Phosphodiesterase Activity Inhibitory Action Test

Compound 1 (Sample 1) was tested for its cyclic AMP phosphodiesterase activity inhibitory action as follows.

To 0.2 mL of 50 mmol/L Tris-HCl buffer solution (pH 7.5) containing 5 mmol/L magnesium chloride, 0.1 mL of 2.5 mg/mL bovine serum albumin solution, 0.1 mL of 0.1 mg/mL cyclic AMP phosphodiesterase solution, and 0.05 mL of the test sample solution (Sample 1, see Table 2 below for final concentration) were added, and the mixture was statically placed at 37° C. for 5 minutes. Then, 0.05 mL of 0.5 mg/mL cyclic AMP solution was added, and the mixture was reacted at 37° C. for 60 minutes. After the reaction was completed, the reaction was stopped by boiling in a boiling water bath for 3 minutes, and then centrifugal separation was applied (2260×g, 10 minutes, 4° C.). Cyclic AMP, a reaction substrate in the supernatant, was analyzed under the following high-performance liquid chromatography conditions. In addition, as a control, the same operation was performed by adding only the solvent to which no sample was added.

### <<High-Performance Liquid Chromatography Conditions>>

Product name: Chromatocorder 12 (available from SYSTEM INSTRUMENTS)
Stationary phase: Wakosil C₁₈-ODS 5 μm (available from FUJIFILM Wako Pure Chemical Corporation)
Column length: 250 mm
Mobile phase: 1 mmol/L TBAP in 25 mmol/L KH₂PO₄: CH3CN=90:10
Mobile phase flow rate: 1.0 mL/min
Detection: 260 nm

Then, a peak area (A) of a cyclic AMP standard product, a peak area (B1) of the supernatant reaction solution of the cyclic AMP standard product and the cyclic AMP phosphodiesterase when no sample was added, and a peak area (B2) of the supernatant reaction solution of the cyclic AMP standard product and the cyclic AMP phosphodiesterase when the test sample was added were obtained. From the obtained results, a decomposition rate (C) of the cyclic AMP standard product when no sample was added and a decomposition rate (D) of the cyclic AMP standard product when the test sample was added were calculated using the following equations.

$$Decomposition rate of standard product without sample addition ( C , \% ) = ( 1 - B 1 / A ) \times 100 \\ Decomposition rate of standard product with test sample addition ( D , \% ) = ( 1 - B 2 / A ) \times 100$$

After that, the cyclic AMP phosphodiesterase activity inhibition rate (%) was calculated from the following equation based on each decomposition rate (C, D) calculated from the above equations.

$$cAMP phosphodiesterase activity inhibition rate ( \% ) = ( 1 - D / C ) \times 100$$

The results are listed in Table 2.

TABLE 2

| | | cAMP phosphodiesterase |
|---|---|---|
| | Concentration | activity inhibition rate |
| Sample No. | (μmol/L) | (%) |
| Sample 1 | 500 | 42.0 |
| | 1000 | 46.8 |
| | 2000 | 55.3 |

As listed in Table 2, Compound 1 (Sample 1) was confirmed to have an excellent cyclic AMP phosphodiesterase activity inhibitory action.

### <Testing Example 2> DPP IV Activity Inhibitory Action Test

Compounds 1 to 3 (Samples 1 to 3) were tested for their dipeptidyl peptidase IV (DPP IV) activity inhibitory actions as follows.

In a 96-well plate, 25 μL of each of test samples (Samples 1 to 3, see Table 3 below for final concentrations) prepared with 25 mM Tris-HCl buffer solution (pH 8.0) and 25 μL of 0.4 μg/mL DPP IV (available from R&D Systems, rhCD26) solution prepared with the above buffer solution were mixed and preincubated at 37° C. for 5 minutes. After that, 50 μL of 0.5 mM Gly-Pro-p-NA-Tos (available from PEPTIDE INSTITUTE, INC.) prepared with the above buffer solution was added, and the mixture was reacted at 37° C. for 90 minutes. After the reaction was completed, the absorbance at a wavelength of 415 nm was measured. From the obtained results, the DPP IV activity inhibition rate (%) was calculated using the following equation.

$$DPP IV acitivity inhibition rate ( \% ) = \{ 1 - ( C - D ) / ( A - B ) \} \times 100$$

Terms in the equation represent the following respective ones.
A: Absorbance at wavelength 415 nm without sample addition and with enzyme addition
B: Absorbance at wavelength 415 nm without sample addition and without enzyme addition
C: Absorbance at wavelength 415 nm with test sample addition and with enzyme addition
D: Absorbance at wavelength 415 nm with test sample addition and without enzyme addition

The results are listed in Table 3.

TABLE 3

| | Concentration | DPP IV activity |
|---|---|---|
| Sample No. | (μmol/L) | inhibition rate (%) |
| Sample 1 | 625 | 7.2 |
| | 2500 | 17.6 |
| | 10000 | 97.1 |
| Sample 2 | 10000 | 96.9 |
| Sample 3 | 2500 | 4.1 |
| | 10000 | 99.8 |

As listed in Table 3, Compound 1 (Sample 1), Compound 2 (Sample 2), and Compound 3 (Sample 3) all exhibited excellent DPP IV inhibitory actions.

10<Testing Example 3> Tyrosinase activity inhibitory action test Compound 3 (Sample 3) was tested for its tyrosinase activity inhibitory action as follows.

In a 48-well plate, 0.2 mL of McIlvaine buffer solution (pH 6.8), 0.06 mL of 0.3 mg/mL tyrosine solution, and 0.18 mL of the test sample (Sample 3, see Table 4 below for final concentration) dissolved in 25% DMSO solution were added, and they were statically placed at 37° C. for 10 minutes. To this, 0.02 mL of 1000 units/mL tyrosinase solution was added, followed by reaction at 37° C. for 15 minutes. After the reaction was completed, the absorbance at a wavelength of 475 nm was measured.

As a blank, the same operation and absorbance measurement were performed in the case in which no enzyme solution was added. Furthermore, as a control, the same measurement was performed in the case in which 25% DMSO solution was added without adding the sample solution. From the obtained measurement results, the tyrosinase activity inhibition rate (%) was calculated using the following equation.

$$Tyrosinase activity inhibition rate ( \% ) = \{ 1 - ( A - B ) / ( C - D ) \} \times 100$$

Terms in the equation represent the following respective ones.
A: Absorbance at wavelength 475 nm with test sample addition and enzyme addition
B: Absorbance at wavelength 475 nm with test sample addition and without enzyme addition
C: Absorbance at wavelength 475 nm without sample addition and with enzyme addition
D: Absorbance at wavelength 475 nm without sample addition and without enzyme addition

The results are listed in Table 4.

TABLE 4

| | Concentration | Tyrosinase activity |
|---|---|---|
| Sample No. | (μmol/L) | inhibition rate (%) |
| Sample 3 | 125 | 4.4 |
| | 250 | 9.6 |
| | 500 | 15.2 |
| | 1000 | 22.8 |

As listed in Table 4, Compound 3 (Sample 3) was found to have an excellent tyrosinase activity inhibitory action.

### <Testing Example 4> Melanin Production Suppressive Action Test on B16 Melanoma Cells

Compounds 1 to 3 (Samples 1 to 3) were tested for their melanin production suppressive actions on B16 melanoma cells as follows.

B16 melanoma cells were cultured using Dulbecco's modified Eagle's medium (DMEM) containing 10% FBS, and then the cells were collected by a trypsin treatment. The collected cells were diluted with DMEM containing 10% FBS and 1 mmol/L theophylline to a cell density of 24.0×10⁴ cells/mL, then seeded with 300 μL per well in a 48-well plate, and cultured for 6 hours.

After the culturing was completed, 300 μL of each of test samples (Samples 1 to 3, see Table 5 below for final concentrations) dissolved in DMEM containing 10% FBS and 1 mmol/L theophylline was added to each well, and culturing was performed for 4 days. As a control, culturing was performed in the same manner using DMEM containing 10% FBS and 1 mmol/L theophylline to which no sample was added. After the culturing was completed, the medium was removed, 200 μL of 2 mol/L NaOH solution was added, the cells were disrupted using an ultrasonic disrupter, and the absorbance at a wavelength of 475 nm was measured. From the measured absorbance value, the amount of melanin was calculated based on a calibration curve created using synthetic melanin (available from SIGMA).

In addition, in order to measure the cell survival rate, after culturing in the same manner as above, the medium was removed, the cells were washed with 400 μL of PBS(−) buffer solution, 200 μL of Neutral Red dissolved at a final concentration of 0.05 mg/mL in DMEM containing 10% FBS was added to each well, and culturing was performed for 2.5 hours. After culturing, the neutral red solution was removed, and 200 μL of ethanol/acetic acid solution (ethanol:acetic acid:water=50:1:49) was added to each well to extract the dye. After extraction, the absorbance at a wavelength of 540 nm was measured. From the obtained results, the melanin production suppression rate (%) corrected by the cell survival rate was calculated using the following equation.

$$Melanin production suppression rate ( \% ) = \{ 1 - ( B / D ) / ( A / C ) \} \times 100$$

Terms in the equation represent the following respective ones.
A: Melanin amount without sample addition
B: Melanin amount with test sample addition
C: Absorbance at wavelength 540 nm without sample addition
D: Absorbance at wavelength 540 nm with test sample addition

The results are listed in Table 5.

TABLE 5

| | | Melanin production |
|---|---|---|
| | Concentration | suppression rate |
| Sample No. | (μmol/L) | (%) |
| Sample 1 | 250 | 63.8 |
| Sample 2 | 62.5 | 8.5 |
| | 250 | 40.2 |
| | 1000 | 69.5 |
| Sample 3 | 250 | 46.2 |
| | 1000 | 57.0 |

As listed in Table 5, Compound 1 (Sample 1), Compound 2 (Sample 2), and Compound 3 (Sample 3) were all recognized to have excellent melanin production suppressive actions.

### <Testing Example 5> Type I Collagen Production Promotive Action Test

Compound 1 (Sample 1) was tested for its type I collagen production promotive action as follows.

Normal human dermal fibroblasts (NB1RGB) were cultured using Dulbecco's modified Eagle's medium (DMEM) containing 10% FBS, and then the cells were collected by a trypsin treatment. The collected cells were diluted with DMEM containing 0.25% FBS to a cell density of 1.6×10⁵ cells/mL, then seeded with 100 μL per well in a 96-well microplate, and cultured overnight.

After the culturing was completed, 100 μL of the test sample (Sample 1, see Table 6 below for final concentration) dissolved in DMEM containing 0.25% FBS was added to each well, and culturing was performed for 3 days. As a control, culturing was performed in the same manner using DMEM containing 0.25% FBS to which no sample was added. After culturing, the amount of type I collagen in the medium of each well was measured by ELISA method. From the measurement results, the type I collagen production promotion rate (%) was calculated using the following equation.

Type I collagen production promotion rate (%)=A/B×100

Terms in the equation represent the following respective ones.
A: Type I collagen amount with test sample addition
B: Type I collagen amount without sample addition

The results are listed in Table 6.

TABLE 6

| | Concentration | Type I collagen production |
|---|---|---|
| Sample No. | (μmol/L) | promotion rate (%) |
| Sample 1 | 15.6 | 107.4 |
| | 62.5 | 112.8 |

As listed in Table 6, Compound 1 (Sample 1) was confirmed to have an excellent type I collagen production promotive action.

### <Testing Example 6> Elastin Production Promotive Action Test

Compound 2 (Sample 2) and Compound 3 (Sample 3) were tested for their elastin production promotive actions as follows.

Normal human dermal fibroblasts (NB1RGB) were cultured using Dulbecco's modified Eagle's medium (DMEM) containing 10% FBS, and then the cells were collected by a trypsin treatment. The collected cells were diluted with the above medium to a cell density of 2.2×10⁵ cells/mL, then seeded with 100 μL per well in a 96-well microplate, and cultured overnight.

After the culturing was completed, the medium was removed, and 150 μL of each of test samples (Samples 2 and 3, see Table 7 below for final concentrations) dissolved in DMEM containing 0.25% FBS was added to each well, and culturing was performed for 5 days. As a control, culturing was performed in the same manner using DMEM containing 0.25% FBS to which no sample was added. After the culturing was completed, the supernatant was recovered, and the amount of elastin released in the culture supernatant was measured by ELISA method. From the measurement results, the elastin production promotion rate (%) was calculated using the following equation.

$$Elastin production promotion rate ( \% ) = A / B \times 100$$

Terms in the equation represent the following respective ones.
A: Elastin amount with test sample addition
B: Elastin amount without sample addition

The results are listed in Table 7.

TABLE 7

| | Concentration | Elastin production |
|---|---|---|
| Sample No. | (μmol/L) | promotion rate (%) |
| Sample 2 | 15.6 | 145.4 |
| | 62.5 | 147.6 |
| | 250 | 189.3 |
| Sample 3 | 62.5 | 174.0 |
| | 250 | 239.0 |

As listed in Table 7, both Compound 2 (Sample 2) and Compound 3 (Sample 3) exhibited excellent elastin production promotive actions.

### <Testing Example 7> Elastase Activity Inhibitory Action Test

Compounds 1 to 3 (Samples 1 to 3) were tested for their elastase activity inhibitory actions as follows.

In a 96-well microplate, 50 μL of each of test samples (Samples 1 to 3, see Table 8 below for final concentrations) prepared with 0.2 mol/L Tris-HCl buffer solution (pH 8.0) and 50 μL of 20 μg/mL elastase type III (available from SIGMA-Aldrich) solution was mixed. After that, 100 μL of 0.4514 mg/mL N-succinyl-Ala-Ala-Ala-p-nitroanilide (available from SIGMA-Aldrich) prepared with the above buffer solution was added, and the mixture was reacted at 25° C. for 15 minutes. After the reaction was completed, the absorbance at a wavelength of 415 nm was measured. In addition, a blank test without enzyme addition was performed in the same manner and correction was made. From the obtained results, the elastase activity inhibition rate (%) was calculated using the following equation.

$$Elastin activity inhibition rate ( \% ) = \{ 1 - ( C - D ) / ( A - B ) \} \times 100$$

Terms in the equation represent the following respective ones.
A: Absorbance at wavelength 415 nm without sample addition and with enzyme addition
B: Absorbance at wavelength 415 nm without sample addition and without enzyme addition
C: Absorbance at wavelength 415 nm with test sample addition and with enzyme addition
D: Absorbance at wavelength 415 nm with test sample addition and without enzyme addition

The results are listed in Table 8.

TABLE 8

| | Concentration | Elastase activity |
|---|---|---|
| Sample No. | (μmol/L) | inhibition rate (%) |
| Sample 1 | 250 | 3.2 |
| | 500 | 6.7 |
| | 1000 | 8.1 |
| Sample 2 | 1000 | 4.2 |
| Sample 3 | 1000 | 5.0 |

As listed in Table 8, Compounds 1 to 3 (Samples 1 to 3) were found to have excellent elastase activity inhibitory actions.

### <Testing Example 8> Hyaluronic Acid Production Promotive Action Test

Compound 1 (Sample 1) and Compound 2 (Sample 2) were tested for their hyaluronic acid production promotive actions as follows.

Normal human dermal fibroblasts (NB1RGB) were cultured using Dulbecco's modified Eagle's medium (DMEM) containing 10% FBS, and then the cells were collected by a trypsin treatment. The collected cells were diluted with DMEM containing 0.25% FBS to a cell density of 1.6×10⁵ cells/mL, then seeded with 100 μL per well in a 96-well plate, and cultured overnight.

After the culturing was completed, 100 μL of each of test samples (Samples 1 and 2, see Table 9 below for final concentrations) dissolved in DMEM containing 0.25% FBS was added to each well, and culturing was performed for 3 days. As a control, culturing was performed in the same manner using DMEM containing 0.25% FBS to which no sample was added. After the culturing was completed, the amount of hyaluronic acid in the medium of each well was measured by a sandwich method using hyaluronic acid binding protein (HABP). From the obtained results, the hyaluronic acid production promotion rate (%) was calculated using the following equation.

Hyaluronic acid production promotion rate (%)=A/B×100

Terms in the equation represent the following respective ones.
A: Hyaluronic acid amount with test sample addition
B: Hyaluronic acid amount without sample addition

The results are listed in Table 9.

TABLE 9

| | Concentration | Hyaluronic acid production |
|---|---|---|
| Sample No. | (μmol/L) | promotion rate (%) |
| Sample 1 | 25 | 124.2 |
| Sample 2 | 50 | 121.8 |
| | 100 | 119.3 |
| | 200 | 111.7 |

As listed in Table 9, Compound 1 (Sample 1) and Compound 2 (Sample 2) were confirmed to have excellent hyaluronic acid production promotive actions.

### <Testing Example 9> Hyaluronic Acid Synthase 3 (HAS3) mRNA Expression Promotive Action Test

Compound 1 (Sample 1) was tested for its HAS3 mRNA expression promotive action as follows.

Normal human neonatal epidermal keratinocytes (NHEK) were precultured using normal human epidermal keratinocyte growth medium (KGM), and the cells were collected by a trypsin treatment. The collected cells were diluted with KGM to a cell density of 15×10⁴ cells/mL, then seeded with 2 mL portions in a 6-well plate (30×10⁴ cells/well), and cultured overnight under conditions of 37° C. and 5% CO₂. After culturing, the medium was replaced with normal human epidermal keratinocyte basal medium (KBM, the above KGM without addition of growth additives (hEGF, BPE, insulin, antibacterial agents, hydrocortisone)), and culturing was further performed for 24 hours.

After culturing for 24 hours, the medium was removed, and 2 mL of the test sample (Sample 1, see Table 10 below for final concentration) dissolved in KBM was added to each well, and culturing was performed under conditions of 37° C. and 5% CO₂ for 24 hours. As a control, culturing was performed in the same manner using KBM to which no sample was added. After culturing, the medium was removed, total RNA was extracted using ISOGEN II (available from NIPPON GENE CO., LTD.), each RNA amount was measured using a spectrophotometer, and the total RNA was prepared so as to be 150 ng/μL.

For HAS3 and GAPDH as an internal standard, the expression levels of mRNA were measured using the above total RNA as a template. Detection was performed through a two-step real-time RT-PCR reaction using a real-time PCR device, Thermal Cycler Dice Real Time System III (available from Takara Bio Inc.), with PrimeScript™ RT Master Mix (Perfect Real Time) (available from Takara Bio Inc.) and TB Green® Fast qPCR Mix (available from Takara Bio Inc.). Primers available from Takara Bio Inc. were used. The expression level of HAS3 mRNA was obtained as a value corrected with a value of GAPDH based on total RNA preparations prepared from cells cultured “with test sample addition” and “without sample addition.” From the obtained values, the HAS3 mRNA expression promotion rate (%) was calculated using the following equation.

HAS3 mRNA expression promotion rate (%)=A/B×100

Terms in the equation represent the following respective ones.
A: Corrected value with test sample addition
B: Corrected value without sample addition

The results are listed in Table 10.

TABLE 10

| | | HAS3 mRNA expression |
|---|---|---|
| Sample No. | Concentration (μmol/L) | promotion rate (%) |
| Sample 1 | 25 | 173. 5 |

As listed in Table 10, Compound 1 (Sample 1) had an excellent HAS3 mRNA expression promotive action.

### <Testing Example 10> Laminin-332 Production Promotive Action Test

Compound 1 (Sample 1) was tested for its laminin-332 production promotive action as follows.

Normal human neonatal epidermal keratinocytes (NHEK) were precultured in a 75 cm² flask using normal human epidermal keratinocyte growth medium (KGM), and the cells were collected by a trypsin treatment. The collected cells were diluted with a medium obtained by removing BPE from KGM (KGM-BPE) to a cell density of 1.0×10⁵ cells/mL, then seeded with 500 μL per well in a 24-well plate, and cultured for 1 day.

After the culturing was completed, the medium was removed, and 500 μL of the test sample (Sample 1, see Table 11 below for final concentration) dissolved in KGM-BPE was added to each well, and culturing was performed for 48 hours. As a control, culturing was performed in the same manner using KGM-BPE to which no sample was added. After the culturing was completed, 100 μL of the medium supernatant was transferred to an ELISA plate and adsorbed to the plate at 37° C. for 2 hours, and then the amount of adsorbed laminin-332 was measured by ELISA method. From the obtained measurement results, the laminin-332 production promotion rate (%) was calculated using the following equation.

Laminin-332 production promotion rate (%)=A/B×100

Terms in the equation represent the following respective ones.
A: Laminin-332 amount with test sample addition
B: Laminin-332 without sample addition

The results are listed in Table 11.

TABLE 11

| | | Laminin-332 production |
|---|---|---|
| Sample No. | Concentration (μmol/L) | promotion rate (%) |
| Sample 1 | 25 | 142. 0 |

As listed in Table 11, Compound 1 (Sample 1) was confirmed to have an excellent laminin-332 production promotive action.

### <Testing Example 11> Epidermal Keratinocyte Proliferation Promotive Action Test

Compound 1 (Sample 1) was tested for its epidermal keratinocyte proliferation promotive action as follows.

Normal human neonatal epidermal keratinocytes (NHEK) were cultured using normal human epidermal keratinocyte growth medium (KGM), and then the cells were collected by a trypsin treatment. The collected cells were diluted with KGM to a cell density of 3.0×10⁴ cells/mL, then seeded with 100 μL per well on a collagen-coated 96-well plate, and cultured overnight. After the culturing was completed, 100 μL of the test sample (Sample 1, see Table 12 below for the final concentration) dissolved in KGM was added to each well, and culturing was performed for 3 days. As a control, culturing was performed in the same manner using KGM to which no sample was added.

The epidermal keratinocyte proliferation promotive action was measured using the MTT assay method. After culturing for 3 days, the medium was removed, and 100 μL of MTT dissolved at a final concentration of 0.4 mg/mL in PBS(−) buffer solution was added to each well. After culturing for 2 hours, blue formazan produced in the cells was extracted with 100 μL of 2-propanol. After extraction, the absorbance at a wavelength of 570 nm was measured. At the same time, the absorbance at a wavelength of 650 nm was measured as turbidity, and the difference between the two was determined as the production amount of blue formazan. From the obtained results, the epidermal keratinocyte proliferation promotion rate (%) was calculated using the following equation.

Epidermal keratinocyte proliferation promotion rate (%)=A/B×100

Terms in the equation represent the following respective ones.
A: Blue formazan production amount with test sample addition
B: Blue formazan production amount without sample addition

The results are listed in Table 12.

TABLE 12

| | | Epidermal keratinocyte |
|---|---|---|
| Sample No. | Concentration (μmol/L) | proliferation promotion rate (%) |
| Sample 1 | 25 | 138.3 |
| | 100 | 152.1 |

As listed in Table 12, Compound 1 (Sample 1) was found to have an excellent epidermal keratinocyte proliferation promotive action.

### <Testing Example 12> ATP Production Promotive Action Test (Epidermal Keratinocytes)

Compound 1 (Sample 1) and Compound 2 (Sample 2) were tested for their ATP production promotive actions as follows.

Normal human neonatal epidermal keratinocytes (NHEK) were cultured using normal human epidermal keratinocyte growth medium (KGM), and then the cells were collected by a trypsin treatment. The collected cells were diluted with KGM to a cell density of 2.0×10⁵ cells/mL, then seeded with 100 μL per well on a collagen-coated 96-well plate, and cultured overnight. After the culturing was completed, the medium was removed, and 100 μL of KGM to which each of test samples (Samples 1 and 2, see Table 13 below for final concentrations) was added was added to each well, and culturing was performed for 2 hours. As a control, culturing was performed in the same manner using KGM to which no sample was added.

The ATP production promotive action was determined by measuring the amount of ATP in cells using the firefly luciferase luminescence method. That is, after culturing for 2 hours, 100 μL of an ATP measurement reagent (available from Toyo B-Net Co., Ltd., trade name “‘Cellular’ ATP measurement reagent”) was added to each well, and chemiluminescence reaction using luciferase was performed. After the reaction, the amount of chemiluminescence proportional to the amount of intracellular ATP was measured using a chemiluminescence measurement device (available from Thermo Fisher Scientific, product name: Varioskan LUX multimode microplate reader). From the obtained results, the ATP production promotion rate (%) was calculated using the following equation.

$$ATP production promotion rate ( \% ) = A / B \times 100$$

Terms in the equation represent the following respective ones.
A: Chemiluminescence amount with test sample addition
B: Chemiluminescence amount without sample addition

The results are listed in Table 13.

TABLE 13

| | | ATP production |
|---|---|---|
| Sample No. | Concentration (μmol/L) | promotion rate (%) |
| Sample 1 | 6.25 | 106.7 |
| Sample 2 | 25 | 112.4 |

As listed in Table 13, Compound 1 (Sample 1) and Compound 2 (Sample 2) were found to have excellent ATP production promotive actions.

### <Testing Example 13> Glutathione Production Promotive Action Test (Dermal Fibroblasts)

Compounds 1 to 3 (Samples 1 to 3) were tested for their glutathione production promotive actions in dermal fibroblasts as follows.

Normal human dermal fibroblasts (NB1RGB) were cultured using α-modified Eagle's minimum essential medium (α-MEM) containing 10% FBS, and then the cells were collected by a trypsin treatment. The collected cells were diluted with α-MEM containing 10% FBS to a cell density of 2.0×10⁵ cells/mL, then seeded with 200 μL per well in a 48-well plate, and cultured for 48 hours.

After culturing, the medium was removed, 200 μL of each of test samples (Samples 1 to 3, see Table 14 below for final concentrations) dissolved in Dulbecco's modified Eagle's medium (DMEM) containing 1% FBS was added to each well, and culturing was further performed for 24 hours. As a control, culturing was performed in the same manner using DMEM containing 1% FBS to which no test sample was added. After the culturing was completed, the medium was removed from each well, and the cells were washed with 400 μL of PBS(−) buffer solution and then lysed using 150 μL of M-PER (available from PIERCE).

Quantitative determination of total glutathione was performed using 100 μL of the lysed cells. That is, 100 μL of lysed cell extract, 50 μL of 0.1 mol/L phosphate buffer solution, 25 μL of 2 mmol/L NADPH, and 25 μL of 3.2 unit/mL glutathione reductase were added to a 96-well plate and heated at 37° C. for 10 minutes, then 25 μL of 10 mmol/L 5,5′-dithiobis(2-nitrobenzoic acid) was added, and the absorbance at a wavelength of 412 nm was measured until 5 minutes later to determine ΔOD/min. The total glutathione concentration was calculated based on a calibration curve created using oxidized glutathione (available from FUJIFILM Wako Pure Chemical Corporation). After correcting the obtained value to the glutathione value per total protein amount, the glutathione production promotion rate (%) was calculated using the following equation.

$$Glutathione production promotion rate ( \% ) = A / B \times 100$$

Terms in the equation represent the following respective ones.
A: Glutathione amount per total protein amount without sample addition
B: Glutathione amount per total protein amount with test sample addition

The results are listed in Table 14.

TABLE 14

| | | Glutathione production |
|---|---|---|
| Sample No. | Concentration (μmol/L) | promotion rate (%) |
| Sample 1 | 62.5 | 125.8 |
| Sample 2 | 1000 | 131.7 |
| Sample 3 | 1000 | 129.9 |

As listed in Table 14, Compound 1 (Sample 1), Compound 2 (Sample 2), and Compound 3 (Sample 3) were all recognized to have excellent glutathione production promotive actions in fibroblasts.

### <Testing Example 14> Transglutaminase-1 (TGM1) mRNA Expression Promotive Action Test

Compounds 1 to 3 (Samples 1 to 3) were tested for their TGM1 mRNA expression promotive actions as follows.

Normal human neonatal epidermal keratinocytes (NHEK) were precultured using normal human epidermal keratinocyte growth medium (KGM), and the cells were collected by a trypsin treatment. The collected cells were diluted with KGM to a cell density of 15×10⁴ cells/mL, then seeded with 2 mL portions in a 6-well plate (30×10⁴ cells/well), and cultured overnight under conditions of 37° C. and 5% CO₂. After culturing, the medium was replaced with normal human epidermal keratinocyte basal medium (KBM, the above KGM without addition of growth additives (hEGF, BPE, insulin, antibacterial agents, hydrocortisone)), and culturing was further performed for 24 hours.

After culturing for 24 hours, the medium was removed, and 2 mL of each of test samples (Samples 1 to 3, see Table 15 below for final concentrations) dissolved in KBM was added to each well, and culturing was performed under conditions of 37° C. and 5% CO₂ for 24 hours. As a control, culturing was performed in the same manner using KBM to which no sample was added. After culturing, the medium was removed, total RNA was extracted using ISOGEN II (available from NIPPON GENE CO., LTD.), each RNA amount was measured using a spectrophotometer, and the total RNA was prepared so as to be 150 ng/μL.

For TGM1 and GAPDH as an internal standard, the expression levels of mRNA were measured using the above total RNA as a template. Detection was performed through a two-step real-time RT-PCR reaction using a real-time PCR device, Thermal Cycler Dice Real Time System III (available from Takara Bio Inc.), with PrimeScript™ RT Master Mix (Perfect Real Time) (available from Takara Bio Inc.) and TB Green® Fast qPCR Mix (available from Takara Bio Inc.). The primers used were those having the sequences 5′-AGGTGGAGCTTAGCCCTGTG-3′ and 5′-GCAAGTGAAGACTGACTCCCTCTC-3′.

The expression level of TGM1 mRNA was obtained as a value corrected with a value of GAPDH based on total RNA preparations prepared from cells cultured “with test sample addition” and “without sample addition.” From the obtained values, the TGM1 mRNA expression promotion rate (%) was calculated using the following equation.

TGM1 mRNA expression promotion rate (%)=A/B×100

Terms in the equation represent the following respective ones.
A: Corrected value with test sample addition
B: Corrected value without sample addition

The results are listed in Table 15.

TABLE 15

| | | TGM1 mRNA expression |
|---|---|---|
| Sample No. | Concentration (μmol/L) | promotion rate (%) |
| Sample 1 | 6.25 | 214.6 |
| | 25 | 528.6 |
| | 100 | 1617.1 |
| Sample 2 | 100 | 160.8 |
| Sample 3 | 25 | 218.5 |
| | 100 | 174.9 |

As listed in Table 15, Compound 1 (Sample 1), Compound 2 (Sample 2), and Compound 3 (Sample 3) all had excellent TGM1 mRNA expression promotive actions.

### <Testing Example 15> Serine Palmitoyltransferase (SPT) mRNA Expression Promotive Action Test

Compound 1 (Sample 1) was tested for its SPT mRNA expression promotive action as follows.

Normal human neonatal epidermal keratinocytes (NHEK) were precultured using normal human epidermal keratinocyte growth medium (KGM), and the cells were collected by a trypsin treatment. The collected cells were diluted with KGM to a cell density of 15×10⁴ cells/mL, then seeded with 2 mL portions in a 6-well plate (30×10⁴ cells/well), and cultured overnight under conditions of 37° C. and 5% CO₂. After culturing, the medium was replaced with normal human epidermal keratinocyte basal medium (KBM, the above KGM without addition of growth additives (hEGF, BPE, insulin, antibacterial agents, hydrocortisone)), and culturing was further performed for 24 hours.

After culturing for 24 hours, the medium was removed, and 2 mL of the test sample (Sample 1, see Table 16 below for final concentration) dissolved in KBM was added to each well, and culturing was performed under conditions of 37° C. and 5% CO₂ for 24 hours. As a control, culturing was performed in the same manner using KBM to which no sample was added. After culturing, the medium was removed, total RNA was extracted using ISOGEN II (available from NIPPON GENE CO., LTD.), each RNA amount was measured using a spectrophotometer, and the total RNA was prepared so as to be 150 ng/μL.

For SPT and GAPDH as an internal standard, the expression levels of mRNA were measured using the above total RNA as a template. Detection was performed through a two-step real-time RT-PCR reaction using a real-time PCR device, Thermal Cycler Dice Real Time System III (available from Takara Bio Inc.), with PrimeScript™ RT Master Mix (Perfect Real Time) (available from Takara Bio Inc.) and TB Green® Fast qPCR Mix (available from Takara Bio Inc.). Primers available from Takara Bio Inc. were used. The expression level of SPT mRNA was obtained as a value corrected with a value of GAPDH based on total RNA preparations prepared from cells cultured “with test sample addition” and “without sample addition.” From the obtained values, the SPT mRNA expression promotion rate (%) was calculated using the following equation.

$$SPT mRNA expression promotion rate ( \% ) = A / B \times 100$$

Terms in the equation represent the following respective ones.
A: Corrected value with test sample addition
B: Corrected value without sample addition

The results are listed in Table 16.

TABLE 16

| | | SPT mRNA expression |
|---|---|---|
| Sample No. | Concentration (μmol/L) | promotion rate (%) |
| Sample 1 | 25 | 114.6 |
| | 100 | 149.9 |

As listed in Table 16, Compound 1 (Sample 1) had an excellent SPT mRNA expression promotive action.

### <Testing Example 16> Aquaporin 3 (AQP3) mRNA Expression Promotive Action Test

Compound 1 (Sample 1) was tested for its AQP3 mRNA expression promotive action as follows.

Normal human neonatal epidermal keratinocytes (NHEK) were precultured using normal human epidermal keratinocyte growth medium (KGM), and the cells were collected by a trypsin treatment. The collected cells were diluted with KGM to a cell density of 15×10⁴ cells/mL, then seeded with 2 mL portions in a 6-well plate (30×10⁴ cells/well), and cultured overnight under conditions of 37° C. and 5% CO₂. After culturing, the medium was replaced with normal human epidermal keratinocyte basal medium (KBM, the above KGM without addition of growth additives (hEGF, BPE, insulin, antibacterial agents, hydrocortisone)), and culturing was further performed for 24 hours.

After culturing for 24 hours, the medium was removed, and 2 mL of the test sample (Sample 1, see Table 17 below for final concentration) dissolved in KBM was added to each well, and culturing was performed under conditions of 37° C. and 5% CO₂ for 24 hours. As a control, culturing was performed in the same manner using KBM to which no sample was added. After culturing, the medium was removed, total RNA was extracted using ISOGEN II (available from NIPPON GENE CO., LTD.), each RNA amount was measured using a spectrophotometer, and the total RNA was prepared so as to be 150 ng/μL.

For AQP3 and GAPDH as an internal standard, the expression levels of mRNA were measured using the above total RNA as a template. Detection was performed through a two-step real-time RT-PCR reaction using a real-time PCR device, Thermal Cycler Dice Real Time System III (available from Takara Bio Inc.), with PrimeScript™ RT Master Mix (Perfect Real Time) (available from Takara Bio Inc.) and TB Green® Fast qPCR Mix (available from Takara Bio Inc.). Primers available from Takara Bio Inc. were used. The expression level of AQP3 mRNA was obtained as a value corrected with a value of GAPDH based on total RNA preparations prepared from cells cultured “with test sample addition” and “without sample addition.” From the obtained values, the AQP3 mRNA expression promotion rate (%) was calculated using the following equation.

$$AQP 3 mRNA expression promotion rate ( \% ) = A / B \times 100$$

Terms in the equation represent the following respective ones.
A: Corrected value with test sample addition
B: Corrected value without sample addition

The results are listed in Table 16.

TABLE 17

| | | AQP3 mRNA expression |
|---|---|---|
| Sample No. | Concentration (μmol/L) | promotion rate (%) |
| Sample 1 | 100 | 207.8 |

As listed in Table 17, Compound 1 (Sample 1) had an excellent AQP3 mRNA expression promotive action.

### <Testing Example 17> Filaggrin (FLG) mRNA Expression Promotive Action Test

Compounds 1 to 3 (Samples 1 to 3) were tested for their FLG mRNA expression promotive actions as follows.

Normal human neonatal epidermal keratinocytes (NHEK) were precultured using normal human epidermal keratinocyte growth medium (KGM), and the cells were collected by a trypsin treatment. The collected cells were diluted with KGM to a cell density of 15×10⁴ cells/mL, then seeded with 2 mL portions in a 6-well plate (30×10⁴ cells/well), and cultured overnight under conditions of 37° C. and 5% CO₂. After culturing, the medium was replaced with normal human epidermal keratinocyte basal medium (KBM, the above KGM without addition of growth additives (hEGF, BPE, insulin, antibacterial agents, hydrocortisone)), and culturing was further performed for 24 hours.

After culturing for 24 hours, the medium was removed, and 2 mL of each of test samples (Samples 1 to 3, see Table 18 below for final concentrations) dissolved in KBM was added to each well, and culturing was performed under conditions of 37° C. and 5% CO₂ for 24 hours. As a control, culturing was performed in the same manner using KBM to which no sample was added. After culturing, the medium was removed, total RNA was extracted using ISOGEN II (available from NIPPON GENE CO., LTD.), each RNA amount was measured using a spectrophotometer, and the total RNA was prepared so as to be 150 ng/μL.

For FLG and GAPDH as an internal standard, the expression levels of mRNA were measured using the above total RNA as a template. Detection was performed through a two-step real-time RT-PCR reaction using a real-time PCR device, Thermal Cycler Dice Real Time System III (available from Takara Bio Inc.), with PrimeScript™ RT Master Mix (Perfect Real Time) (available from Takara Bio Inc.) and TB Green® Fast qPCR Mix (available from Takara Bio Inc.). Primers available from Takara Bio Inc. were used. The expression level of FLG mRNA was obtained as a value corrected with a value of GAPDH based on total RNA preparations prepared from cells cultured “with test sample addition” and “without sample addition.” From the obtained values, the FLG mRNA expression promotion rate (%) was calculated using the following equation.

$$FLG mRNA expression promotion rate ( \% ) = A / B \times 100$$

Terms in the equation represent the following respective ones.
A: Corrected value with test sample addition
B: Corrected value without sample addition

The results are listed in Table 18.

TABLE 18

| | | FLG mRNA expression |
|---|---|---|
| Sample No. | Concentration (μmol/L) | promotion rate (%) |
| Sample 1 | 25 | 197.2 |
| | 100 | 342.0 |
| Sample 2 | 25 | 168.2 |
| | 100 | 129.8 |
| Sample 3 | 25 | 196.6 |

As listed in Table 18, Compound 1 (Sample 1), Compound 2 (Sample 2), and Compound 3 (Sample 3) all had excellent FLG mRNA expression promotive actions.

### <Testing Example 18> Claudin-1 (CLDN1) mRNA Expression Promotive Action Test

Compound 1 (Sample 1) and Compound 3 (Sample 3) were tested for their CLDN1 mRNA expression promotive actions as follows.

Normal human neonatal epidermal keratinocytes (NHEK) were precultured using normal human epidermal keratinocyte growth medium (KGM), and the cells were collected by a trypsin treatment. The collected cells were diluted with KGM to a cell density of 15×10⁴ cells/mL, then seeded with 2 mL portions in a 6-well plate (30×10⁴ cells/well), and cultured overnight under conditions of 37° C. and 5% CO₂. After culturing, the medium was replaced with normal human epidermal keratinocyte basal medium (KBM, the above KGM without addition of growth additives (hEGF, BPE, insulin, antibacterial agents, hydrocortisone)), and culturing was further performed for 24 hours.

After culturing for 24 hours, the medium was removed, and 2 mL of each of test samples (Samples 1 and 3, see Table 19 below for final concentrations) dissolved in KBM was added to each well, and culturing was performed under conditions of 37° C. and 5% CO₂ for 24 hours. As a control, culturing was performed in the same manner using KBM to which no sample was added. After culturing, the medium was removed, total RNA was extracted using ISOGEN II (available from NIPPON GENE CO., LTD.), each RNA amount was measured using a spectrophotometer, and the total RNA was prepared so as to be 150 ng/μL.

For CLDN1 and GAPDH as an internal standard, the expression levels of mRNA were measured using the above total RNA as a template. Detection was performed through a two-step real-time RT-PCR reaction using a real-time PCR device, Thermal Cycler Dice Real Time System III (available from Takara Bio Inc.), with PrimeScript™ RT Master Mix (Perfect Real Time) (available from Takara Bio Inc.) and TB Green® Fast qPCR Mix (available from Takara Bio Inc.). Primers available from Takara Bio Inc. were used. The expression level of CLDN1 mRNA was obtained as a value corrected with a value of GAPDH based on total RNA preparations prepared from cells cultured “with test sample addition” and “without sample addition.” From the obtained values, the CLDN1 mRNA expression promotion rate (%) was calculated using the following equation.

$$CLDN 1 mRNA expression promotion rate ( \% ) = A / B \times 100$$

Terms in the equation represent the following respective ones.
A: Corrected value with test sample addition
B: Corrected value without sample addition

The results are listed in Table 19.

TABLE 19

| | | CLDN1 mRNA expression |
|---|---|---|
| Sample No. | Concentration (μmol/L) | promotion rate (%) |
| Sample 1 | 25 | 256.6 |
| | 100 | 568.6 |
| Sample 3 | 25 | 125.5 |

As listed in Table 19, both Compound 1 (Sample 1) and Compound 3 (Sample 3) had excellent CLDN1 mRNA expression promotive actions.

### <Testing Example 19> Claudin-4 (CLDN4) mRNA Expression Promotive Action Test

Compound 1 (Sample 1) was tested for its CLDN4 mRNA expression promotive action as follows.

Normal human neonatal epidermal keratinocytes (NHEK) were precultured using normal human epidermal keratinocyte growth medium (KGM), and the cells were collected by a trypsin treatment. The collected cells were diluted with KGM to a cell density of 15×10⁴ cells/mL, then seeded with 2 mL portions in a 6-well plate (30×10⁴ cells/well), and cultured overnight under conditions of 37° C. and 5% CO₂. After culturing, the medium was replaced with normal human epidermal keratinocyte basal medium (KBM, the above KGM without addition of growth additives (hEGF, BPE, insulin, antibacterial agents, hydrocortisone)), and culturing was further performed for 24 hours.

After culturing for 24 hours, the medium was removed, and 2 mL of the test sample (Sample 1, see Table 20 below for final concentrations) dissolved in KBM was added to each well, and culturing was performed under conditions of 37° C. and 5% CO₂ for 24 hours. As a control, culturing was performed in the same manner using KBM to which no sample was added. After culturing, the medium was removed, total RNA was extracted using ISOGEN II (available from NIPPON GENE CO., LTD.), each RNA amount was measured using a spectrophotometer, and the total RNA was prepared so as to be 150 ng/μL.

For CLDN4 and GAPDH as an internal standard, the expression levels of mRNA were measured using the above total RNA as a template. Detection was performed through a two-step real-time RT-PCR reaction using a real-time PCR device, Thermal Cycler Dice Real Time System III (available from Takara Bio Inc.), with PrimeScript™ RT Master Mix (Perfect Real Time) (available from Takara Bio Inc.) and TB Green® Fast qPCR Mix (available from Takara Bio Inc.). Primers available from Takara Bio Inc. were used. The expression level of CLDN4 mRNA was obtained as a value corrected with a value of GAPDH based on total RNA preparations prepared from cells cultured “with test sample addition” and “without sample addition.” From the obtained values, the CLDN4 mRNA expression promotion rate (%) was calculated using the following equation.

$$CLDN 4 mRNA expression promotion rate ( \% ) = A / B \times 100$$

Terms in the equation represent the following respective ones.
A: Corrected value with test sample addition
B: Corrected value without sample addition

The results are listed in Table 20.

TABLE 20

| | | CLDN4 mRNA expression |
|---|---|---|
| Sample No. | Concentration (μmol/L) | promotion rate (%) |
| Sample 1 | 100 | 489.7 |

As listed in Table 20, Compound 1 (Sample 1) had an excellent CLDN1 mRNA expression promotive action.

### <Testing Example 20> Occludin (OCLN) mRNA Expression Promotive Action Test

Compound 1 (Sample 1) and Compound 3 (Sample 3) were tested for their OCLN mRNA expression promotive actions as follows.

Normal human neonatal epidermal keratinocytes (NHEK) were precultured using normal human epidermal keratinocyte growth medium (KGM), and the cells were collected by a trypsin treatment. The collected cells were diluted with KGM to a cell density of 15×10⁴ cells/mL, then seeded with 2 mL portions in a 6-well plate (30×10⁴ cells/well), and cultured overnight under conditions of 37° C. and 5% CO₂. After culturing, the medium was replaced with normal human epidermal keratinocyte basal medium (KBM, the above KGM without addition of growth additives (hEGF, BPE, insulin, antibacterial agents, hydrocortisone)), and culturing was further performed for 24 hours.

After culturing for 24 hours, the medium was removed, and 2 mL of each of test samples (Samples 1 and 3, see Table 20 below for final concentrations) dissolved in KBM was added to each well, and culturing was performed under conditions of 37° C. and 5% CO₂ for 24 hours. As a control, culturing was performed in the same manner using KBM to which no sample was added. After culturing, the medium was removed, total RNA was extracted using ISOGEN II (available from NIPPON GENE CO., LTD.), each RNA amount was measured using a spectrophotometer, and the total RNA was prepared so as to be 150 ng/μL.

For OCLN and GAPDH as an internal standard, the expression levels of mRNA were measured using the above total RNA as a template. Detection was performed through a two-step real-time RT-PCR reaction using a real-time PCR device, Thermal Cycler Dice Real Time System III (available from Takara Bio Inc.), with PrimeScript™ RT Master Mix (Perfect Real Time) (available from Takara Bio Inc.) and TB Green® Fast qPCR Mix (available from Takara Bio Inc.). Primers available from Takara Bio Inc. were used. The expression level of OCLN mRNA was obtained as a value corrected with a value of GAPDH based on total RNA preparations prepared from cells cultured “with test sample addition” and “without sample addition.” From the obtained values, the OCLN mRNA expression promotion rate (%) was calculated using the following equation.

$$OCLN mRNA expression promotion rate ( \% ) = A / B \times 100$$

Terms in the equation represent the following respective ones.
A: Corrected value with test sample addition
B: Corrected value without sample addition

The results are listed in Table 21.

TABLE 21

| | | OCLN mRNA expression |
|---|---|---|
| Sample No. | Concentration (μmol/L) | promotion rate (%) |
| Sample 1 | 25 | 209.9 |
| | 100 | 557.3 |
| Sample 3 | 25 | 141.5 |

As listed in Table 21, both Compound 1 (Sample 1) and Compound 3 (Sample 3) had excellent OCLN mRNA expression promotive actions.

### <Testing Example 21> Advanced Glycation End Product (AGEs) Formation Suppressive Action Test

Compound 1 (Sample 1) and Compound 2 (Sample 2) were tested for their AGEs formation suppressive actions as follows.

In a 96-well type I collagen-coated plate (available from AGC Inc.), 100 μL of a mixed solution of 0.2M D(−)-ribose prepared with PBS(−) buffer solution and each of test samples (Samples 1 and 2, see Table 22 below for final concentrations) were added and statically placed at 37° C. for 20 days to form AGEs. In addition, PBS(−) buffer solution only as a negative control and a 0.2M D(−)-ribose solution prepared with PBS(−) buffer solution as a positive control were statically placed in the same manner. After 20 days, the amount of AGEs was measured by the ELISA method using an anti-AGEs antibody (available from TPANS GENIC INC.), and the AGEs formation suppressive action was evaluated. From the obtained results, the AGEs formation suppression rate (%) was calculated using the following equation.

$$AGEs formation suppression rate ( \% ) = \{ ( B - C ) / ( B - A ) \} \times 100$$

Terms in the equation represent the following respective ones.
A: Absorbance at wavelength 405 nm in negative control
B: Absorbance at wavelength 405 nm in positive control
C: Absorbance at wavelength 405 nm with test sample addition

The results are listed in Table 22.

TABLE 22

| | | AGEs formation |
|---|---|---|
| Sample No. | Concentration (μmol/L) | suppression rate (%) |
| Sample 1 | 125 | 45.7 |
| | 500 | 69.4 |
| | 2000 | 78.4 |
| Sample 2 | 2000 | 13.3 |

As listed in Table 22, Compound 1 (Sample 1) and Compound 2 (Sample 2) exhibited excellent AGEs formation suppressive actions.

### <Testing Example 22> Advanced Glycation End Product (AGEs) Decomposition Promotive Action Test

Compound 1 (Sample 1) was tested for its AGEs decomposition promotive action as follows.

In a 96-well type I collagen-coated plate (available from AGC Inc.), 100 μL of 0.2M D(−)-ribose solution prepared with PBS(−) buffer solution was added and statically placed at 37° C. for 2 weeks to form AGEs. In addition, the plate in which only the PBS(−) buffer solution was added was statically placed as a negative control in the same manner. After 2 weeks, 100 μL of the test sample (Sample 1, see Table 23 below for final concentration) prepared with PBS (−) buffer solution was added to each of the plates and statically further placed at 37° C. for 20 days. As a positive control, only PBS(−) buffer solution was statically placed in the same manner as substitute for the test sample, and as a negative control, only PBS(−) buffer solution was continued to be statically placed in the same manner. After 20 days, the amount of AGEs was measured by the ELISA method using an anti-AGEs antibody (available from TPANS GENIC INC.), and the AGEs decomposition promotive action was evaluated. From the obtained results, the AGEs decomposition promotion rate (%) was calculated using the following equation.

$$AGEs decomposition promotion rate ( \% ) = \{ ( B - C ) / ( B - A ) \} \times 100$$

Terms in the equation represent the following respective ones.
A: Absorbance at wavelength 405 nm in negative control
B: Absorbance at wavelength 405 nm in positive control
C: Absorbance at wavelength 405 nm with test sample addition

The results are listed in Table 23.

TABLE 23

| | | AGEs decomposition |
|---|---|---|
| Sample No. | Concentration (μmol/L) | promotion rate (%) |
| Sample 1 | 125 | 37.7 |
| | 500 | 39.9 |
| | 2000 | 34.1 |

As listed in Table 23, Compound 1 (Sample 1) exhibited an excellent AGEs decomposition promotive action.

### <Testing Example 23> Testosterone 5α-Reductase Inhibitory Action Test

Compounds 1 to 3 (Samples 1 to 3) were tested for their testosterone 5α-reductase inhibitory actions as follows.

In a V-bottom test tube with a lid, 20 μL of a 4.2 mg/mL testosterone (available from FUJIFILM Wako Pure Chemical Corporation) solution prepared with propylene glycol and 825 μL of 5 mmol/L Tris-HCl (pH 7.13) buffer solution containing 1 mg/mL NADPH were mixed.

Furthermore, 80 μL of each of test sample solutions (Samples 1 to 3, see Table 24 below for final concentrations) prepared with 80% ethanol and 75 μL of S-9 (available from Oriental Yeast Co., Ltd., rat liver homogenate) were added, mixed, and incubated at 37° C. for 60 minutes. After that, 1 mL of methylene chloride was added to stop the reaction. This was centrifuged (1600×g, 10 minutes), the methylene chloride layer was separated, and the separated methylene chloride layer was subjected to gas chromatography analysis under the following conditions to determine the concentrations of 3α-androstanediol, 5α-dihydrotestosterone (5α-DHT), and testosterone. As a control, the same process was performed using the same amount (80 μL) of the sample solvent in place of the test sample solution, and the control was subjected to gas chromatography analysis.

### <<Gas Chromatography Conditions>>

Device used: Shimadzu GC-2010 (available from Shimadzu Corporation)
Column: DB-1701 (inner diameter: 0.53 mm, length: 30 m, film thickness: 1.0 μm) (available from J&W Scientific)
Column temperature: 240° C.
Inlet temperature: 300° C.
Detector: FID
Sample injection amount: 1 μL
Split ratio: 1:2
Carrier gas: Nitrogen gas
Carrier gas flow rate: 12 mL/min

Quantitative determination of the concentrations of 3α-androstanediol, 5α-DHT, and testosterone was performed by the following method.

Standard products of 3α-androstanediol, 5α-DHT, and testosterone were each dissolved in ethanol, and the solutions were subjected to gas chromatography analysis. From the concentrations (μg/mL) and peak areas of these compounds, the correspondence relationship between the peak area and the concentration of each compound was preliminarily obtained. Then, the concentration per peak area of each of 3α-androstanediol, 5α-DHT, and testosterone after the reaction between testosterone and S-9 was obtained based on the following equation (1) using the correspondence relationship which was preliminarily obtained.

$$A = B \times C / D … \qquad ( 1 )$$

Terms in the equation represent the following respective ones.
A: Concentration of 3α-androstanediol, 5α-DHT, or testosterone
B: Peak area of 3α-androstanediol, 5α-DHT, or testosterone
C: Concentration of standard product
D: Peak area of standard product

Using the compound concentration calculated based on the equation (1), the conversion ratio (concentration ratio between the concentration of 3α-androstanediol and 5α-DHT produced by reduction of testosterone due to testosterone 5α-reductase and the initial concentration of testosterone) was calculated based on the following equation (2).

$$Conversion ratio = ( E + F ) / ( E + F + G ) … \qquad ( 2 )$$

Terms in the equation represent the following respective ones.
E: Concentration of 3α-androstanediol (μg/mL)
F: Concentration of 5α-DHT (μg/mL)
G: Concentration of testosterone (μg/mL)

Using the conversion ratio calculated based on the equation (2), the testosterone 5α-reductase inhibition rate (%) was calculated based on the following equation (3).

$$Testosterone 5 α - reductase inhibition rate ( \% ) = ( 1 - H / I ) \times 100 … \qquad ( 3 )$$

Terms in the equation represent the following respective ones.
H: Conversion ratio with test sample addition
I: Conversion ratio without sample addition

The results are listed in Table 24.

TABLE 24

| | | Testosterone 5 α-reductase |
|---|---|---|
| Sample No. | Concentration (μmol/L) | inhibition rate (%) |
| Sample 1 | 2000 | 34.3 |
| | 8000 | 100 |
| Sample 2 | 2000 | 71.0 |
| | 8000 | 100 |
| Sample 3 | 2000 | 76.4 |
| | 8000 | 100 |

As listed in Table 24, Compound 1 (Sample 1), Compound 2 (Sample 2), and Compound 3 (Sample 3) were confirmed to have excellent testosterone 5α-reductase inhibitory actions.

### <Testing Example 24> Follicle Dermal Papilla Cell Proliferation Promotive Action Test

Compound 1 (Sample 1) was tested for its follicle dermal papilla cell proliferation promotive action as follows.

Normal human hair follicle dermal papilla cells (HFDPC, derived from the parietal region of a male) were cultured using follicle dermal papilla cell growth medium (PCGM, available from TOYOBO CO., LTD.) containing 1% FCS and growth additives, and then the cells were collected by a trypsin treatment. The collected cells were diluted to a cell density of 1.0×10⁴ cells/mL using Dulbecco's modified Eagle's medium (DMEM) containing 10% FBS, then seeded with 200 μL per well in a collagen-coated 96-well plate, and cultured for 3 days.

After that, the medium was removed, 200 μL of the test sample (Sample 1, see Table 25 below for final concentration) dissolved in serum-free DMEM was added to each well, and culturing was further performed for 4 days. As a control, culturing was performed in the same manner using serum-free DMEM to which no sample was added. After the culturing was completed, the follicle dermal papilla cell proliferation promotive action was measured by MTT assay. That is, the medium was removed, 100 μL of 0.4 mg/mL MTT prepared with serum-free DMEM was added to each well, culturing was further performed for 2 hours, and then blue formazan produced in the cells was extracted with 100 μL of 2-propanol. The absorbance of this extract was measured at 570 nm giving the maximum absorption point of blue formazan. At the same time, the absorbance at a wavelength of 650 nm was measured as turbidity, and the difference between the two was determined as the production amount of blue formazan. From the measurement results, the follicle dermal papilla cell proliferation promotion rate (%) was calculated based on the following equation.

$$Follicle dermal papilla cell proliferation promotion rate ( \% ) = A / B \times 100$$

Terms in the equation represent the following respective ones.
A: Blue formazan production amount with test sample addition
B: Blue formazan production amount without sample addition

The results are listed in Table 25.

TABLE 25

| | | Follicle dermal papilla cell |
|---|---|---|
| | Concentration | proliferation promotion rate |
| Sample No. | (μmol/L) | (%) |
| Sample 1 | 100 | 124.8 |

As listed in Table 25, Compound 1 (Sample 1) was found to have an excellent follicle dermal papilla cell proliferation promotive action.

### <Testing Example 25> Nitric Oxide (NO) Production Suppressive Action Test

Compound 1 (Sample 1) was tested for its nitric oxide (NO) production suppressive action as follows.

Mouse macrophage cells (RAW264.7) were cultured using Dulbecco's modified Eagle's medium (DMEM) containing 10% FBS, and then the cells were collected using a cell scraper. The collected cells were diluted with phenol red-free DMEM containing 10% FBS to a cell density of 3.0×10⁶ cells/mL, then seeded with 100 μL per well in a 96-well plate, and cultured for 4 hours.

After culturing, the medium was removed, 100 μL of the test sample (Sample 1, see Table 26 below for final concentration) dissolved in phenol red-free DMEM containing DMSO at a final concentration of 0.5% and 10% FBS was added to each well, 100 μL of lipopolysaccharide (LPS, final concentration 1 μg/mL, E. coli 0111:B4, available from DIFCO) dissolved in phenol red-free DMEM containing 10% FBS was added, and culturing was performed for 48 hours. As a control, the LPS process was performed in the same manner using, as substitute for the test sample solution, phenol red-free DMEM containing DMSO at a final concentration of 0.5% and 10% FBS to which no sample was added.

The production amount of nitric oxide (NO) was measured using the amount of nitrite ions (NO²⁻) as an index. After the culturing was completed, the same amount of Griess reagent (5 mass % phosphoric acid solution containing 1 mass % sulfanilamide and 0.1 mass % N-1-naphthyl ethylendiamine dihydrochloride) as the culture supernatant was added to the culture solution in each well and reacted for 10 minutes at room temperature. After the reaction, the absorbance at a wavelength of 540 nm was measured. The nitric oxide (NO) production suppression rate (%) was calculated by the following equation based on the nitric oxide (NO) production amount when no sample was added (control).

$$NO production suppression rate ( \% ) = \{ ( B - A ) / B \} \times 100$$

Terms in the equation represent the following respective ones.
A: NO amount with test sample addition
B: NO amount with test sample addition

The results are listed in Table 26.

TABLE 26

| | | NO production |
|---|---|---|
| | Concentration | suppression rate |
| Sample No. | (μmol/L) | (%) |
| Sample 1 | 31.2 | 14.9 |
| | 125 | 49.4 |
| | 500 | 77.8 |

As listed in Table 26, Compound 1 (Sample 1) was confirmed to have an excellent nitric oxide production suppressive action.

### <Testing Example 26> Hyaluronidase Activity Inhibitory Action Test

Compounds 1 to 3 (Samples 1 to 3) were tested for their hyaluronidase activity inhibitory actions as follows.

To 0.2 mL of each of test samples (Samples 1 to 3, see Table 27 below for final concentrations) dissolved in 0.1 mol/L acetate buffer solution (pH 3.5), 0.1 mL of hyaluronidase solution (available from SIGMA, Type IV-S, from bovine tests, 400 NF units/mL) was added, and the mixture was statically placed at 37° C. for 20 minutes. Furthermore, 0.2 mL of 2.5 mmol/L calcium chloride was added as an activator, and the mixture was statically placed at 37° C. for 20 minutes. To this was added 0.5 mL of 0.8 mg/mL sodium hyaluronate solution (from rooster comb), and the mixture was reacted at 37° C. for 40 minutes. After that, 0.2 mL of 0.4 mol/L sodium hydroxide was added to stop the reaction, it was cooled, and then 0.2 mL of boric acid solution was added to each reaction solution, which was boiled for 3 minutes. After cooling on ice, 6 mL of p-DABA reagent was added and reacted at 37° C. for 20 minutes. After that, the absorbance at a wavelength of 585 nm was measured.

In addition, as a blank, the same operation and absorbance measurement were performed in the case in which no enzyme solution was added. Furthermore, as a control, the same measurement was performed in the case in which distilled water was added without adding the sample solution. From the obtained results, the hyaluronidase activity inhibition rate (%) was calculated using the following equation.

$$Hyaluronidase activity inhibition rate ( \% ) = \{ 1 - ( A - B ) / ( C - D ) \} \times 100$$

Terms in the equation represent the following respective ones.
A: Absorbance at wavelength 585 nm with test sample addition and with enzyme addition
B: Absorbance at wavelength 585 nm with test sample addition and without enzyme addition
C: Absorbance at wavelength 585 nm without sample addition and with enzyme addition
D: Absorbance at wavelength 585 nm without sample addition and without enzyme addition

The results are listed in Table 27.

TABLE 27

| | | Hyaluronidase activity |
|---|---|---|
| | Concentration | inhibition rate |
| Sample No. | (μmol/L) | (%) |
| Sample 1 | 250 | 4.9 |
| | 1000 | 19.9 |
| Sample 2 | 1000 | 15.8 |
| Sample 3 | 62.5 | 16.5 |
| | 250 | 20.2 |
| | 1000 | 41.1 |

As listed in Table 27, Compound 1 (Sample 1), Compound 2 (Sample 2), and Compound 3 (Sample 3) were all confirmed to have excellent hyaluronidase activity inhibitory actions.

### <Testing Example 27> Hexosaminidase Release Suppressive Action Test

Compound 2 (Sample 2) was tested for its hexosaminidase release suppressive action as follows.

Rat basophil leukemia cells (RBL-2H3) were cultured using spinner modified Eagle's minimum essential medium (S-MEM) containing 15% FBS, and then the cells were collected by a trypsin treatment. The collected cells were diluted with S-MEM containing 15% FBS to a cell density of 4.0×10³ cells/mL, DNP-specific IgE was added to a final concentration of 0.5 μg/mL, and the cells were seeded with 100 μL per well in a 96-well plate and cultured overnight.

After culturing, the medium was removed and the cells were washed twice with 100 μL of Shiraganian buffer solution. Then, 30 μL of the buffer solution and 10 μL of the test sample (Sample 2, see Table 28 below for the final concentration) dissolved in the buffer solution were added to each well and statically placed at 37° C. for 10 minutes. As a control, the same operation was performed using 40 μL of Shiragarian buffer solution without addition of the sample. Subsequently, 10 μL of 400 ng/mL DNP-BSA solution was added, and the mixture was statically placed at 37° C. for 15 minutes to release hexosaminidase.

After that, the release was stopped by statically placing the 96-well plate on ice. In a new 96-well plate, 10 μL of the cell supernatant from each well was collected, 10 μL of 1 mmol/L p-nitrophenyl-N-acetyl-β-D-glucosaminide (p-NAG) solution was added to each well, and the mixture was reacted at 37° C. for 1 hour.

After the reaction was completed, 250 μL of 0.1 mol/L Na₂CO₃/NaHCO₃ was added to each well, the absorbance at wavelengths of 415 nm and 650 nm was measured, and the value obtained by subtracting the absorbance at 650 nm from the absorbance at 415 nm was adopted as the corrected value. From the obtained measurement results, the hexosaminidase release suppression rate (%) was calculated using the following equation.

$$Hexosaminidase release suppression rate ( \% ) = \{ 1 - ( B / A ) \} \times 100$$

Terms in the equation represent the following respective ones.
A: Absorbance at wavelength 415-650 nm without sample addition
B: Absorbance at wavelength 415-650 nm with test sample addition

The results are listed in Table 28.

TABLE 28

| | | Hexosaminidase release |
|---|---|---|
| | Concentration | suppression rate |
| Sample No. | (μmol/L) | (%) |
| Sample 2 | 500 | 19.3 |

As listed in Table 28, Compound 2 (Sample 2) was confirmed to have an excellent hexosaminidase release suppressive action.

### <Testing Example 28> Prostaglandin E₂ (PGE₂) Production Suppressive Action Test in Mouse Macrophages

Compound 1 (Sample 1) was tested for its PGE₂ production suppressive action as follows.

Mouse macrophage cells (RAW264.7) were cultured using Dulbecco's modified Eagle's medium (DMEM) containing 10% FBS, and then the cells were collected using a cell scraper. The collected cells were diluted with DMEM containing 10% FBS to a concentration of 2.0×10⁵ cells/mL, then seeded with 100 μL per well in a 96-well plate, and cultured for 18 hours.

After the culturing was completed, the medium was replaced with a medium containing 500 μmol/L aspirin and cultured for 4 hours in order to acetylate and inactivate the already existing COX-1 and the small amount of COX-2 expressed. After that, the cells were washed three times with PBS(−) buffer solution, 100 μL of the test sample (Sample 1, see Table 29 below for final concentration) dissolved in DMEM containing DMSO at a final concentration of 0.5% and 10% FBS was added to each well, then 100 μL of lipopolysaccharide (LPS) (available from DIFCO, E. coli 0111; B4) dissolved at a final concentration of 1 μg/mL in DMEM containing 10% FBS, and the cells were cultured for 16 hours. As a control, culturing was performed in the same manner using DMEM containing DMSO at a final concentration of 0.5% and 10% FBS to which no sample was added. After the culturing was completed, the amount of prostaglandin E₂ in the culture supernatant of each well was determined using PGE₂ EIA Kit (available from Cayman Chemical). From the obtained results, the PGE₂ production suppression rate (%) was calculated using the following equation.

$${PGE}_{2} production suppression rate ( \% ) = \{ 1 - ( A - C ) / ( B - C ) \} \times 100$$

Terms in the equation represent the following respective ones.
A: PGE₂ amount with test sample addition and with LPS stimulation
B: PGE₂ amount without sample addition and with LPS stimulation
C: PGE₂ amount without sample addition and without LPS stimulation

The results are listed in Table 29.

TABLE 29

| | | PGE₂ production |
|---|---|---|
| | Concentration | suppression rate |
| Sample No. | (μmol/L) | (%) |
| Sample 1 | 31.25 | 17.9 |
| | 125 | 17.7 |
| | 500 | 41.3 |

As listed in Table 29, Compound 1 (Sample 1) was confirmed to have an excellent PGE₂ production suppressive action in macrophages.

### <Testing Example 29> Glutathione Production Promotive Action Test (Hepatocytes)

Compounds 1 to 3 (Samples 1 to 3) were tested for their glutathione production promotive actions in hepatocytes as follows.

Normal human liver cells (hepatocytes) were cultured using Dulbecco's modified Eagle's medium (DMEM) containing 10% FBS, and then the cells were collected by a trypsin treatment. The collected cells were diluted with DMEM containing 10% FBS to a cell density of 10×10⁴ cells/mL, then seeded with 200 μL per well in a 48-well plate, and cultured overnight.

After culturing, the medium was removed, 200 μL of each of test samples (Samples 1 to 3, see Table 30 below for final concentrations) dissolved in DMEM containing 1% FBS was added to each well, and culturing was further performed for 24 hours. As a control, culturing was performed in the same manner using DMEM containing 1% FBS to which no sample was added. After the culturing was completed, the medium was removed from each well, and the cells were washed with 400 μL of PBS(−) and then lysed using 150 μL of M-PER (available from PIERCE).

Quantitative determination of total glutathione was performed using 100 μL of the lysed cells. That is, 100 μL of lysed cell extract, 50 μL of 0.1 mol/L phosphate buffer solution, 25 μL of 2 mmol/L NADPH, and 25 μL of 3.2 unit/mL glutathione reductase were added to a 96-well plate and heated at 37° C. for 10 minutes, then 25 μL of 10 mmol/L 5,5′-dithiobis(2-nitrobenzoic acid) was added, and the absorbance at a wavelength of 412 nm was measured until 5 minutes later to determine ΔOD/min. The total glutathione concentration was calculated based on a calibration curve created using oxidized glutathione (available from FUJIFILM Wako Pure Chemical Corporation). After correcting the obtained value to the glutathione value per total protein amount, the glutathione production promotion rate (%) was calculated based on the following equation.

$$Glutathione production promotion rate ( \% ) = B / A \times 100$$

Terms in the equation represent the following respective ones.
A: Glutathione amount per total protein amount without sample addition
B: Glutathione amount per total protein amount with test sample addition

The results are listed in Table 30.

TABLE 30

| | | Glutathione production |
|---|---|---|
| | Concentration | promotion rate |
| Sample No. | (μmol/L) | (%) |
| Sample 1 | 31.2 | 123.9 |
| | 125 | 124.8 |
| | 500 | 113.4 |
| Sample 2 | 31.2 | 119.2 |
| | 125 | 118.1 |
| Sample 3 | 31.2 | 133.9 |
| | 125 | 124.6 |
| | 500 | 119.2 |

As listed in Table 30, Compound 1 (Sample 1), Compound 2 (Sample 2), and Compound 3 (Sample 3) were all recognized to have excellent glutathione production promotive actions in hepatocytes.

### <Testing Example 30> ATP Production Promotive Action Test in Hepatocytes

Compounds 1 to 3 (Samples 1 to 3) were tested for their ATP production promotive actions in hepatocytes as follows.

Normal human liver cells (hepatocytes) were cultured using Dulbecco's modified Eagle's medium (DMEM) containing 10% FBS, and then the cells were collected by a trypsin treatment. The collected cells were diluted with DMEM containing 10% FBS to a cell density of 2.0×10⁵ cells/mL, then seeded with 100 μL per well in a 96-well plate, and cultured overnight.

After the culturing was completed, the medium was removed, 100 μL of each of test samples (Samples 1 to 3, see Table 31 below for final concentrations) dissolved in DMEM containing 10% FBS was added to each well, and culturing was performed for 2 hours. As a control, culturing was performed in the same manner using DMEM containing 10% FBS to which no sample was added.

The ATP production promotive action was evaluated by measuring the amount of ATP in cells using the firefly luciferase luminescence method. That is, after culturing for 2 hours, 100 μL of an ATP measurement reagent (available from Toyo B-Net Co., Ltd., trade name “‘Cellular’ ATP measurement reagent”) was added to each well, and chemiluminescence reaction using luciferase was performed. After the reaction, the amount of chemiluminescence proportional to the amount of intracellular ATP was measured using a chemiluminescence measurement device (available from Thermo Fisher Scientific, product name: Varioskan LUX multimode microplate reader). From the obtained results, the ATP production promotion rate (%) was calculated using the following equation.

$$ATP production promotion rate ( \% ) = A / B \times 100$$

Terms in the equation represent the following respective ones.
A: Chemiluminescence amount with test sample addition
B: Chemiluminescence amount without sample addition

The results are listed in Table 31.

TABLE 31

| | | ATP production |
|---|---|---|
| | Concentration | promotion rate |
| Sample No. | (μmol/L) | (%) |
| Sample 1 | 250 | 128.4 |
| | 500 | 166.8 |
| | 1000 | 168.9 |
| Sample 2 | 250 | 141.9 |
| | 500 | 159.1 |
| | 1000 | 165.1 |
| Sample 3 | 250 | 128.0 |
| | 500 | 136.5 |
| | 1000 | 156.1 |

As listed in Table 31, Compound 1 (Sample 1), Compound 2 (Sample 2), and Compound 3 (Sample 3) were all recognized to have excellent ATP production promotive actions in hepatocytes.

### Compounding Example 1

Tablets having the following composition were produced using an ordinary method.

| | | |
|---|---|---|
| Compound 1 | 5.0 | mg |
| Dolomite (containing 20% calcium and 10% magnesium) | 83.4 | mg |
| Casein phosphopeptide | 16.7 | mg |
| Vitamin C | 33.4 | mg |
| Maltitol | 136.8 | mg |
| Collagen | 12.7 | mg |
| Sucrose fatty acid ester | 12.0 | mg |

### Compounding Example 2

An oral liquid preparation having the following composition was produced using an ordinary method.

<<Composition in 1 Ampule (100 mL)>>

| | |
|---|---|
| Compound 2 | 0.3 mass % |
| Sorvit | 12.0 mass % |
| Sodium benzoate | 0.1 mass % |
| Flavor | 1.0 mass % |
| Calcium sulfate | 0.5 mass % |
| Purified water | the balance |
| | (100 mass %) |

### Compounding Example 3

Encapsulated formulation having the following composition was produced using an ordinary method. No. 1 hard gelatin capsules were used as the capsules.

<<Composition in 1 Capsule (1 Tablet 200 mg)>>

| | | |
|---|---|---|
| Compound 3 | 10.0 | mg |
| Cornstarch | 70.0 | mg |
| Lactose | 100.0 | mg |
| Calcium lactate | 10.0 | mg |
| Hydroxypropylcellulose (HPC-L) | 10.0 | mg |

### Compounding Example 4

A milky lotion was produced using an ordinary method according to the following composition.

| | |
|---|---|
| Compound 1 | 0.01 g |
| Jojoba oil | 4.00 g |
| 1,3-butylene glycol | 3.00 g |
| Arbutin | 3.00 g |
| Polyoxyethylene cetyl ether (20E.O.) | 2.50 g |
| Olive oil | 2.00 g |
| Squalane | 2.00 g |
| Cetanol | 2.00 g |
| Glyceryl monostearate | 2.00 g |
| Polyoxyethylene sorbitan oleate (20E.O.) | 2.00 g |
| Methyl parahydroxybenzoate | 0.15 g |
| Stearyl glycyrrhetinate | 0.10 g |
| Engelhardtia chrysolepis extract | 0.10 g |
| Dipotassium glycyrrhizate | 0.10 g |
| Ginkgo biloba leaf extract | 0.10 g |
| Conchiolin | 0.10 g |
| Phellodendron amurense extract | 0.10 g |
| Chamomile extract | 0.10 g |
| Fragrance | 0.05 g |
| Purified water | The balance |
| | (total amount 100 g) |

### Compounding Example 5

A cream having the following composition was produced using an ordinary method.

| | | |
|---|---|---|
| Compound 2 | 0.05 | g |
| Sophora root extract | 0.1 | g |
| Baikal skullcap extract | 0.1 | g |
| Liquid paraffin | 5.0 | g |
| White beeswax | 4.0 | g |
| Squalane | 10.0 | g |
| Cetanol | 3.0 | g |
| Lanolin | 2.0 | g |
| Stearic acid | 1.0 | g |
| Polyoxyethylene sorbitan oleate (20E.O.) | 1.5 | g |
| Glyceryl monostearate | 3.0 | g |
| Oil-soluble licorice extract | 0.1 | g |
| 1,3-butylene glycol | 6.0 | g |
| Methyl parahydroxybenzoate | 1.5 | g |
| Fragrance | 0.1 | g |

| | |
|---|---|
| Purified water | The balance |
| | (total amount 100 g) |

### Compounding Example 6

A beauty serum having the following composition was produced using an ordinary method.

| | | |
|---|---|---|
| Compound 3 | 0.01 | g |
| Chamomile extract | 0.1 | g |
| Ginseng extract | 0.1 | g |
| Xanthan gum | 0.3 | g |
| Hydroxyethyl cellulose | 0.1 | g |
| Carboxy vinyl polymer | 0.1 | g |
| 1,3-butylene glycol | 4.0 | g |
| Dipotassium glycyrrhizate | 0.1 | g |
| Glycerin | 2.0 | g |
| Potassium hydroxide | 0.25 | g |
| Fragrance | 0.01 | g |
| Preservative (methyl parahydroxybenzoate) | 0.15 | g |
| Ethanol | 2.0 | g |

| | |
|---|---|
| Purified water | The balance |
| | (total amount 100 g) |

### Compounding Example 7

A hair tonic having the following composition was produced using an ordinary method.

| | |
|---|---|
| Compound 1 | 0.2 g |
| Compound 2 | 0.1 g |
| Compound 3 | 0.1 g |
| Tocopheryl acetate | Appropriate amount |
| Cepharanthin | 0.002 g |
| Isopropyl methylphenol | 0.1 g |
| Sodium hyaluronate | 0.15 g |
| Glycerin | 15.0 g |
| Ethanol | 15.0 g |
| Fragrances | Appropriate amount |
| Chelate agent (sodium edetate) | Appropriate amount |
| Preservative (Hinokitiol) | Appropriate amount |
| Solubilizer (polyoxyethylene | Appropriate amount |
| cetyl ether) | |
| Purified water | The balance |
| | (total amount 100 g) |

### Compounding Example 8

A shampoo having the following composition was produced using an ordinary method.

| | | |
|---|---|---|
| Compound 1 | 0.2 | g |
| Compound 2 | 0.2 | g |
| Compound 3 | 0.2 | g |
| Marjoram extract | 1.0 | g |
| Plum fruit extract | 0.2 | g |
| Sodium methyl cocoyl taurate | 10.0 | g |
| Cocamidopropyl Betaine | 10.0 | g |
| Polyoxyethylene alkyl ether sodium sulfate | 20.0 | g |
| Coconut oil fatty acid diethanolamide | 4.0 | g |
| Propylene glycol | 2.0 | g |

| | |
|---|---|
| Fragrances | Appropriate amount |
| Purified water | The balance |
| | (total amount 100 g) |

1-9. (canceled)

10. A method for preventing or ameliorating metabolic syndrome in a patient in need of preventing or ameliorating metabolic syndrome, comprising steps of:
preparing an anti-metabolic syndrome agent comprising one or more active ingredients selected from the group consisting of compounds 1 to 3 represented by General Formula (I) below, and
administering an effective amount of the anti-metabolic syndrome agent to the patient for preventing or ameliorating metabolic syndrome.

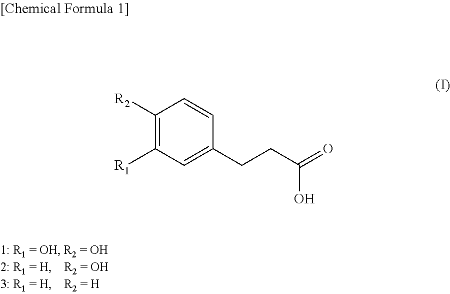

11. The method for preventing or ameliorating metabolic syndrome according to claim 10, wherein the anti-metabolic syndrome agent prevents or ameliorates the metabolic syndrome by inhibiting a cAMP phosphodiesterase activity and/or inhibiting DPP IV activity.

12. The method for preventing or ameliorating metabolic syndrome according to claim 10, wherein the anti-metabolic syndrome agent is an oral composition.

13. The method for preventing or ameliorating metabolic syndrome according to claim 10, wherein the anti-metabolic syndrome agent is a cosmetic.
